(19) United States
(12) Patent Application Publication
(10) Pub. No.: US 2024/0379214 A1
(43) Pub. Date: Nov. 14, 2024

(54) DIRECTIONAL ALTERATION OF MICROBIOME COMPOSITION THROUGH DIET MODIFICATION

(71) Applicant: Zoe Limited, London (GB)

(72) Inventors: Jonathan Thomas Wolf, London (GB); Richard James Davies, London (GB); Francesca Giordano, London (GB)

(73) Assignee: Zoe Limited, London (GB)

(21) Appl. No.: 18/691,196

(22) PCT Filed: Sep. 21, 2022
(86) PCT No.: PCT/EP2022/076241

§ 371 Date: Mar. 12, 2024

Related U.S. Application Data
(60) Provisional application No. 63/247,268, filed on Sep. 22, 2021.

(51) Int. Cl. G16H 20/60

(52) U.S. Cl. CPC G16H 20/60

(57) ABSTRACT

Techniques are disclosed for using food guidance, such as personalized food guidance, to alter the composition of the microbiome of an individual. Such alteration may include increasing the presence, abundance, or relative abundance of a pro-health indicator microbe and/or decreasing the presence, abundance, or relative abundance of a poor health indicator microbe.

### CROSS-REFERENCE TO RELATED APPLICATIONS

This application is the 371 National Phase of International Application No. PCT/EP2022/076241, filed on Sep. 21, 2022, which claims priority to and the benefit of the earlier filing date of U.S. Provisional Application No. 63/247,268, filed on Sep. 22, 2021, which is incorporated by reference herein in its entirety (including Exhibits A-D of that application).

### FIELD OF THE DISCLOSURE

The present disclosure relates generally to systems and methods to alter microbiome composition, for instance based on diet modification.

### BACKGROUND OF THE DISCLOSURE

Dietary contributions to health, and particularly to long-term chronic conditions such as obesity, metabolic syndrome, and cardiac events, are of universal importance. This is especially true as obesity and associated mortality and morbidity have risen dramatically over the past decades and continue to do so worldwide. The reasons for this relatively rapid change have remained unclear, with the gut microbiome implicated as one of several potentially causal human-environmental interactions (Brown & Hazen, Nat. Rev. Microbiol. 16:171-181, 2018; Mozaffarian, Circulation 133:187-225, 2016; Musso et al., Annu. Rev. Med. 62, 361-380, 2011; Le Chatelier et al., Nature 500:541-546, 2013). Surprisingly, the details of the microbiome's role in obesity and cardiometabolic health have proven difficult to define reproducibly in large, diverse human populations-contrary to their behavior in mice-likely due to the complexity of habitual diets, the difficulty of measuring them at scale, and the highly personalized nature of the microbiome (Gilbert et al., Nat. Med. 24:392-400, 2018).

Today, individuals can measure a large number of health characteristics without having to go to a lab or clinic. For example, individuals may obtain an analysis of their microbiome by mailing a sample, collected at home, to a company for analysis. Generally, a microbiome analysis includes determining the composition and function of a community of microbes in a particular location, such as within the gut of an individual. A microbiome of the gut is made up of trillions of microorganisms, such as bacteria, and their genetic material that live in the intestinal tract, including bacteria, archaea or archaebacteria, viruses, and microeukaryotes.

These microorganisms appear to be an important part of digesting food, assisting with absorbing and synthesizing nutrients, regulating metabolism, body weight, and immune regulation, as well as contributing to regulating brain functions and mood. Microbiomes of different individuals, however, vary greatly. For instance, it is estimated that only ten to thirty percent of the bacterial species in a microbiome is common across different individuals. Much of this diversity of microbiomes remains unexplained, yet diet, environment, and host genetics appear to play a part. Determining how to utilize the results of the microbiome analysis, including how to intentionally alter the presence, absence, and/or abundance of individual microbial members in a microbiome, can be challenging.

Obesity represents a major global health challenge with its prevalence in adults and children increasing worldwide. Currently, effective interventions to prevent or reverse obesity have, for the most part, failed to be translated into successful personalized strategies that are likely to produce sustained healthy behavior. A number of different factors make intentional weight loss difficult and facilitate weight regain after weight loss. In addition to decreases in resting energy expenditure, increases in appetite while dieting can make it difficult for an individual to lose weight.

Further, providing guidance to individuals about what to eat and how to change a diet can be very difficult and confusing. Not only do individuals have a large variety of food choices, but food that is healthy for one individual may not be healthy for another individual. Age, sex, weight, the microbiome of an individual, as well as other factors affect what foods an individual should select to eat. For example, while low carbohydrate food or low-fat food may be beneficial for one individual, that same low carbohydrate or low-fat food choice may not be beneficial for another individual.

### SUMMARY OF THE DISCLOSURE

Provided herein are methods and systems for altering the microbiome composition of a subject, such as a human subject, through diet modification.

Disclosed herein the first demonstration the inventors are aware of whereby dietary guidance is shown to provide a material (and positive) change to the microbiome of subjects when the dietary guidance is adhered to.

In a described embodiment, correlations between food consumed and microbiome members (“bugs”) for thousands of people were used to identify foods that are to be promoted (e.g., those that tend to support a healthy condition in the subject, or to move a subject more toward a healthy state) and foods that are to be demoted (e.g., those that do not support a healthy condition, or that tend to support an unhealthy condition in the subject, or to move a subject more toward a less healthy state). When a subject/individual is advised (based on these correlations) which foods to promote and which to demote in their diet, as part of a food guidance or diet program, and the subject then adheres to this food guidance program over a period of time, the subject's/individual's gut health (and metabolic health) is improved.

This improvement in gut health may include an increase in the presence or abundance or relative abundance of at least one pro-health microbe in the subject's/individual's microbiome, or a decrease in the presence or abundance or relative abundance of at least one poor health microbe in the subject's/individual's microbiome, or some combination of both.

This disclosure provides methods of altering the presence, absence, and/or relative or absolute quantity of member(s) of a gut microbiome in a subject, by altering the diet (e.g., pattern and/or content of food and beverages consumed) of the subject. Broadly speaking, the subject's diet is altered by providing the subject with a diet or food guidance program that is different from their current intake regimen, which program is then adhered to by the subject for a period of time. The content of the subject's microbiome is assessed at least once, for instance, before the diet or food guidance program is implemented; by measuring before, the microbiome fingerprint of the subject can be used as factor(s) that influence the program itself. Optionally, the microbiome is assayed more than once, for instance a second time that is some period after initiation of adherence to the food guidance program. This latter microbiome fingerprint can be used to assess the success (in improving gut health) of the food guidance program, for instance.

Also provided is a reiterative method, which allows for continued improvement over time. In these reiterative embodiments, measurement of change in a microbiome fingerprint from one sample timepoint to the next is used in order to improve generalized or personalized food guidance to that individual. In examples of such methods, how many “good” and/or “bad” bugs have changed between the two time points (and other changes in the fingerprint like diversity, etc.) is measured, and this is with correlate food guidance provided to the individual over time. Such correlations can be improved, for instance, by tracking what the individual consumes-including as an aspect of tracking their adherence to the food guidance program more generally. Machine learning can be used to compare the data gathered from a single subject/individual with similar data gathered from a databank of other people (for instance, thousands of other people), also following particular food guidance and for whom measurements in changes in microbiome diversity and content are available at at least two time points. The comparison is then used to revise and improve the dietary guidance (such as personalized dietary guidance) that is given to the individual, and optionally to other individuals, so as to improve on the levels of decrease in bad bugs (and increase in good bugs). It is also contemplated that in some embodiments, the individual's updated/reiterated diet program is influenced by the current and prior record of their own results and changes, rather than relying on comparison to a database of results from other individuals.

In the methods, systems, and uses described herein, the food guidance program that is conveyed to a single individual (also referred to herein as a subject) is prepared at least in part based on or in reference to a database of biomarkers and other information related to, for instance, general health, gut health, microbiome content (such as presence, absence, abundance, or relative abundance of specific microbes, and/or overall diversity, and other recognized measures of microbiome health), nutritional information, circadian rhythm, gathered from a plurality of individuals. Methods to prepare such an aggregated database of information are known, including methods developed by Zoe Limited. Additional guidance regarding representative methods to generate a database useful in preparing food guidance for a group or an individual (e.g., a personalized food guidance) may be found for instance in: WO 2019/155436 “Generating Predicted Values of Biomarkers For Scoring Food”; WO 2019/155437 “Generating Personalized Nutritional Recommendations Using Predicted Values Of Biomarkers”; WO 2019/224308 “Improving the Accuracy of Measuring Nutritional Responses in a Non-Clinical Setting”; WO 2020/043702 “Generating Personalized Food Recommendations from Different Food Sources”; WO 2020/043705 “Improving The Accuracy of Test Data Outside the Clinic”; WO 2020/043706 “Using at Home Measures to Predict Clinical State and Improving the Accuracy of At Home Measurements/Predictions Data Associated with Circadian Rhythm and Meal Timing”; WO 2021/038530 “Generalized Personalized Food Guidance Using Predicted Food Responses”; US 2021-0065873 A1 “Generating Personalized Food Guidance Using Predicted Food Responses”; and WO 2021/186047 A1 “Microbiome Fingerprints, Dietary Fingerprints, and Microbiome Ancestry, and Methods of their Use”.

The current disclosure provides in exemplar embodiments use of a diet program (which is optionally a personalized diet program, personalized for the individual subject, or personalized to a group to which the subject belongs) to improve the gut microbiome of a subject, where the improvement to the gut microbiome includes: increasing presence or relative abundance of at least one pro-health indicator microbe in the microbiome of the subject; decreasing presence or relative abundance of at least one poor health indicator microbe in the microbiome of the subject; or both. By way of example, the diet program may be prepared at least in part based on: presence, absence, or relative abundance of at least one pro-health indicator microbe in the microbiome of the subject; and/or presence, absence, or relative abundance of at least one poor health indicator microbe in the microbiome of the subject.

Also provided are methods that include: preparing a food guidance program (which is optionally a personalized food guidance program, personalized for the individual subject, or personalized to a group to which the subject belongs) for a subject; communicating the food guidance program to the subject; wherein, when the subject follows the food guidance program: presence or relative abundance of at least one pro-health indicator microbe in the microbiome of the subject is increased; presence or relative abundance of at least one poor health indicator microbe in the microbiome of the subject is decreased; or both.

Yet another embodiment is a method that includes: detecting in a microbiome sample from the human subject one or more pro-health indicator microbes selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and modifying the diet of the human using a food guidance program (which is optionally a personalized food guidance program, personalized for the individual subject, or personalized to a group to which the subject belongs) that increases growth or survival of the pro-health indicator microbe(s); and/or detecting in a microbiome sample from the human subject one or more poor health indicator microbe selected from the group consisting of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and modifying the diet of the human using a food guidance program (which is optionally a personalized food guidance program, personalized for the individual subject, or personalized to a group to which the subject belongs) that decreases growth or survival of the poor health indicator microbe(s).

Also provided are methods of altering abundance of at least one microbial species in a gut microbiome of a subject, including: providing the subject with a personalized food guidance program, which personalized food guidance program is developed based at least in part on a food score personalized for the subject, and modifying food intake of the subject based on the personalized food guidance plan, thereby altering the abundance of at least one microbial species in the gut microbiome of the subject. For instance, in some examples of this method embodiment, altering abundance includes: increasing presence or relative abundance of at least one pro-health indicator microbe in the microbiome of the subject; decreasing presence or relative abundance of at least one poor health indicator microbe in the microbiome of the subject; or both.

Yet another embodiment is a method of improving gut microbiome balance/health of a subject, including developing a personalized food guidance program for the subject, communicating the personalized food guidance program to the subject, modifying food intake of the subject, in accordance with the personalized food guidance program, thereby improving gut microbiome balance/health of the subject.

Also described are methods of improving a gut microbiome profile of a subject, which methods include: assaying the gut microbiome of the subject, thereby producing a first microbiome signature of the subject; developing a personalized food guidance program for the subject, which personalized food guidance program will improve the gut microbiome of the subject when the subject follows the program; communicating the personalized food guidance program to the subject; assaying the gut microbiome of the subject at a time the after the subject begins to follow the customized diet guidance plan, thereby producing a second microbiome signature of the subject; comparing the first microbiome signature of the subject to the second microbiome signature of the subject, wherein the first and second microbiome signatures include the presence, absence, or relative abundance of one or more pro-health indicator microbes and/or the presence, absence, or relative abundance of one or more poor health indicator microbes; and wherein an increase in the presence or relative abundance of the one or more pro-health indicator microbes and/or a decrease in the presence or relative abundance of the one or more poor health indicator microbes from the first signature to the second signature constitutes an improved gut microbiome profile of the subject.

In any of the described embodiments, the personalized food guidance program is based at least based in part on one or more of: a non-microbial biomarker of the subject; a nutritional response of the subject; medical history of the subject; a health condition of the subject; predicted hunger of the subject; predicted response to food consumption of the subject; glucose response of the subject; fat response of the subject; microbiome data of the subject; data about the subject's overall health; and/or potential health risks for the subject.

In examples of each of the use and method embodiments, the pro-health indicator microbe is selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and/or the poor health indicator microbe is selected from the group including Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli

For instance, in some specific uses and methods, the pro-health indicator microbe(s) includes Prevotella copri. Alternatively, or in addition, in some uses and methods the poor health indicator microbe(s) includes one or more of Collinsella intestinalis, Clostridium sp CAG 58, Blautia hydrogenotrophica, Flavonifractor plautii, Clostridium leptum, and/or Ruthenibacterium lactatiformans.

In any of the use and method embodiments, the food guidance program may be developed based at least in part based on or in reference to a database including one or more of: correlations between food consumed and microbiome members for thousands of individual subjects; and/or a plurality of foods designated as “to be promoted in a diet”, wherein this designation indicates the food tends to support a healthy condition in the subject, or tends to move a subject incorporating the food in their diet more toward a healthy state; and/or a plurality of foods designated as “to be demoted in a diet”, wherein this designation indicates the food tends to not support a healthy condition, or tend to support an unhealthy condition in the subject, or tends to move a subject incorporating the food in their diet more toward a less healthy state.

Also provided in another embodiment is use of a diet program, such as a personalized diet program, to improve the gut microbiome of a subject or to alter the abundance of at least one microbial species in a gut microbiome of a subject, essentially as disclosed herein.

Yet another embodiment is a method that includes: preparing a food guidance program for a subject; communicating the food guidance program to the subject; wherein, when the subject follows the food guidance program: presence or relative abundance of at least one pro-health indicator microbe in the microbiome of the subject is increased, wherein the one or more pro-health indicator microbes are selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; or presence or relative abundance of at least one poor health indicator microbe in the microbiome of the subject is decreased, wherein the one or more poor health indicator microbe selected from the group consisting of Clostridium sp CAG 58, Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Blautia hydrogenotrophica, Anaerotruncus colihominis, Ruminococcus gnavus, Flavonifractor plautii, Clostridium leptum, Ruthenibacterium lactatiformans, and Escherichia coli; or both.

### BRIEF DESCRIPTION OF THE DRAWINGS

FIG. 1 is a block diagram depicting an illustrative operating environment in which microbiome data is analyzed to generate microbiome fingerprints, dietary fingerprints, and microbiome ancestry for users.

FIG. 2 is a block diagram depicting an illustrative operating environment in which a data ingestion service receives, and processes test data associated with at home tests and sample collections.

FIG. 3 is a flow diagram showing a process illustrating aspects of a mechanism disclosed herein for obtaining and utilizing microbiome data for a user to generate microbiome fingerprints, dietary fingerprints, and microbiome ancestry for users.

FIG. 4 is a flow diagram showing a process illustrating aspects of a mechanism disclosed herein for generating a microbiome fingerprint for a user.

FIG. 5 is a flow diagram showing a process illustrating aspects of a mechanism disclosed herein for generating a dietary fingerprint for a user.

FIG. 6 is a flow diagram showing a process illustrating aspects of a mechanism disclosed herein for generating a microbiome ancestry for a user.

FIG. 7 is a flow diagram showing a process illustrating aspects of a mechanism disclosed herein for obtaining test data, including microbiome data, that may be utilized for generating microbiome fingerprints, dietary fingerprints, and microbiome ancestry for users.

FIG. 8 is a computer architecture diagram showing one illustrative computer hardware architecture for implementing a computing device that might be utilized to implement aspects of the various examples presented herein.

FIG. 9 is a pair of graphs illustrating participant's weight change in kg between the initial test and the retest (Retest weight-Initial weight), versus Adherence to the program (left graph) and versus the level of diet change consequence of the program (right graph).

FIG. 10 is a graph showing the relative abundance of the 15 “good bugs” for the Initial Cohort (first in each pair) and the Retest Cohort (second in each pair). The small white rectangular boxes represent the cohort mean relative abundance for a microbe. The * indicate the species with a statistically significant change in their relative abundance (adjusted p-value≤0.2).

FIG. 11 is a graph showing the relative abundance of the 15 “bad bugs” for the Initial Cohort (first in each pair) and the Retest Cohort (second in each pair). The small white rectangular boxes represent the cohort mean relative abundance for a microbe. The * indicate the species with a statistically significant change in their relative abundance (adjusted p-value≤0.2).

FIG. 12 is a schematic illustration of the METHOD Control Arm of a described Randomized Control Trial Study, further described in Example 2.

FIG. 13 is a schematic illustration of the METHOD Intervention Arm of a described Randomized Control Trial Study, further described in Example 2.

### DETAILED DESCRIPTION

Aspects of the current disclosure are now described with additional details and options. Any headings included in this document do not limit the interpretation of the disclosure and are provided for organizational purposes only.

Methods are provided that employ correlations between food/beverage consumed and microbiome members (“bugs”) for thousands of people to identify foods that are to be promoted and foods that are to be demoted. By way of example, foods to be promoted in a diet may include those that tend to support a healthy condition in the subject, or to move a subject more toward a healthy state. By way of example, foods to be demoted in a diet may include e.g., those that do not support a healthy condition, or that tend to support an unhealthy condition in the subject, or to move a subject more toward a less healthy state. When a subject/individual is advised (based on these correlations) which foods to promote and which to demote in their diet, as part of a food guidance or diet program, and the subject then adheres to this food guidance program over a period of time, the subject's/individual's gut health (and metabolic health) is improved.

The food guidance advice provided to the individual may be generalized—in that it is applicable to more than one subject, or a group or category of subjects (which group or category can be defined in various ways)—or it may be personalized, in that the advice takes into account one or more factors that are specific to the subject to whom/which the food guidance is provided. For instance, a microbiome fingerprint from the individual may be used to personalize the food guidance by taking into account the initial or current state of the individual's microbiome.

This improvement in gut health may include an increase in the presence or abundance or relative abundance of at least one pro-health microbe in the subject's/individual's microbiome, or a decrease in the presence or abundance or relative abundance of at least one poor health microbe in the subject's/individual's microbiome, or some combination of both.

This disclosure provides methods of altering the presence, absence, and/or relative or absolute quantity of member(s) of a gut microbiome in a subject, by altering the diet (e.g., pattern and/or content of food and beverages consumed) of the subject. Broadly speaking, the subject's diet is altered by providing the subject with a diet or food guidance program that is different from their current intake regimen, which program is then adhered to by the subject for a period of time. The content of the subject's microbiome is assessed at least once, for instance, before the diet or food guidance program is implemented; by measuring before, the microbiome fingerprint of the subject can be used as factor(s) that influence the program itself. Optionally, the microbiome is assayed more than once, for instance a second time that is some period after initiation of adherence to the food guidance program. This latter microbiome fingerprint can be used to assess the success (in improving gut health) of the food guidance program, for instance.

Also provided is a reiterative method, which allows for continued improvement over time. In these reiterative embodiments, measurement of change in a microbiome fingerprint from one sample timepoint to the next is used in order to improve generalized or personalized food guidance to that individual. In examples of such methods, how many “good” and/or “bad” bugs have changed between the two time points (and other changes in the fingerprint like diversity, etc.) is measured, and this is with correlate food guidance provided to the individual over time. Such correlations can be improved, for instance, by tracking what the individual consumes-including as an aspect of tracking their adherence to the food guidance program more generally. Machine learning can be used to compare the data gathered from a single subject/individual with similar data gathered from a databank of other people (for instance, thousands of other people), also following particular food guidance and for whom measurements in changes in microbiome diversity and content are available at at least two time points. The comparison is then used to revise and improve the dietary guidance (such as personalized dietary guidance) that is given to the individual, and optionally to other individuals, so as to improve on the levels of decrease in bad bugs (and increase in good bugs). It is also contemplated that in some embodiments, the individual's updated/reiterated diet program is influenced by the current and prior record of their own results and changes, rather than relying on comparison to a database of results from other individuals.

Alternatively, instead of providing food guidance that is generalized for a group of individuals, a nutritional service considers one or more factors of an individual and based on those factor(s) develops personalized (that is, personalized to an individual subject or a group of subjects) food guidance.

Factors that may be considered when developing a (personalized) food guidance or food guidance program include, but are not limited to: predicted hunger of an individual, the individuals predicted responses to food consumption, glucose responses, fat responses, microbiome data, data about the individual's overall health, potential health risks for the individual, and/or other data associated with the individual to generate the food guidance. According to some examples, the individual's glucose responses and/or the responses of other individuals to glucose, and other data, are used to generate the food guidance.

In some configurations, the food guidance may include a hunger score that predicts a hunger level of an individual at a time (or for more than one time) after the individual has or is planning to consume food.

In the methods, systems, and uses described herein, the food guidance program that is conveyed to a single individual (also referred to herein as a subject) is prepared at least in part based on or in reference to a database of biomarkers and other information related to, for instance, general health, gut health, microbiome content (such as presence, absence, abundance, or relative abundance of specific microbes, and/or overall diversity, and other recognized measures of microbiome health), nutritional information, circadian rhythm, gathered from a plurality of individuals. Methods to prepare such an aggregated database of information are known, including methods developed by Zoe Limited. Additional guidance regarding representative methods to generate food guidance of a group or an individual (e.g., a personalized food guidance) may be found for instance in: WO 2019/155436 “Generating Predicted Values of Biomarkers For Scoring Food”; WO 2019/155437 “Generating Personalized Nutritional Recommendations Using Predicted Values Of Biomarkers”; WO 2019/224308 “Improving the Accuracy of Measuring Nutritional Responses in a Non-Clinical Setting”; WO 2020/043702 “Generating Personalized Food Recommendations from Different Food Sources”; WO 2020/043705 “Improving The Accuracy of Test Data Outside the Clinic”; WO 2020/043706 “Using at Home Measures to Predict Clinical State and Improving the Accuracy of At Home Measurements/Predictions Data Associated with Circadian Rhythm and Meal Timing”; WO 2021/038530 “Generalized Personalized Food Guidance Using Predicted Food Responses”; and US 2021-0065873 A1 “Generating Personalized Food Guidance Using Predicted Food Responses”.

### Representative Methods for Generation, Collection, & Analysis of Microbiome Data.

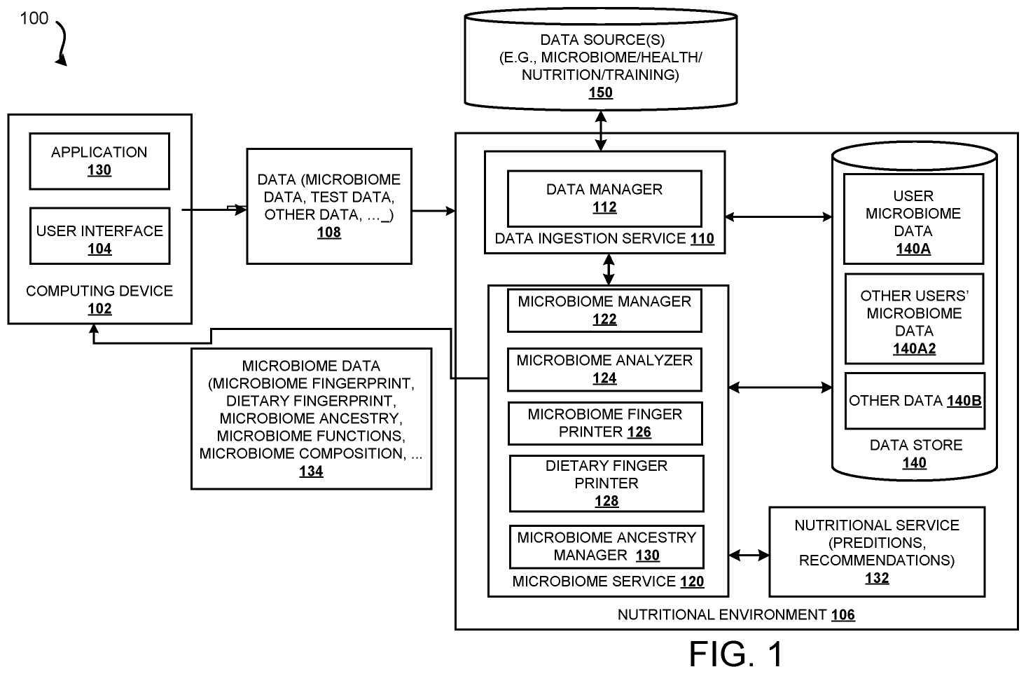

FIG. 1 is a block diagram depicting an illustrative operating environment 100 in which microbiome data is analyzed to generate microbiome fingerprints, dietary fingerprints, and microbiome ancestry for users. An individual, such as an individual interested in obtaining microbiome fingerprints, dietary fingerprints, and microbiome ancestry information, may communicate with the nutritional environment 106 using a computing device 102 and possibly other computing devices, such as mobile electronic devices.

In some configurations, an individual may generate and provide data 108, such as microbiome data, test data, and/or other data. According to some examples, the user may utilize a variety of at home biological collection devices, which collect a biological sample. These devices may include but are not limited to “At Home Blood Tests” which use blood extraction devices such as finger pricks which in some examples are used with dried blood spot cards, button operated blood collection devices using small needles and vacuum to collect liquid capillary blood and the like. In some examples there may be home biological collection devices such as a stool test which is then assayed to produce biomarker test data such as gut microbiome data. As exemplified herein, the subject from which the biological sample is obtained may be a human subject. Other animal subjects are also contemplated, including non-human primates, companion animals, domestic animals, livestock, endangered and threatened animals, laboratory animals, and so forth.

A computing device, such as a mobile phone or a tablet computing device can also be used to improve the accuracy of the measurements. For instance, instead of relying on an individual to accurately record the time a test was taken or a sample was obtained, the computing device 102 can record information that is associated with the event. The computing device 102 may also be utilized to capture the timing data associated with the test (e.g., the time the test was performed, . . . ), or the sample was collected, and provide that data to a data ingestion service 110. As an example, a clock (or some other timing device) of the computing device 102 may be used to record the time the measurement(s) were collected and/or samples were obtained.

As illustrated in FIG. 1, the operating environment 100 includes one or more computing devices 102, in communication with a nutritional environment 106. In some examples, the nutritional environment 106 may be associated with and/or implemented by resources provided by a service provider network such as provided by a cloud computing company. The nutritional environment 106 includes a data ingestion service 110, a microbiome service 120, a nutritional service 132, and a data store 140. The nutritional service 132 can be utilized to generate personalized nutritional recommendations. For example, the personalized nutritional recommendations can be generated using techniques described in U.S. Patent Publication No. US 2019-0252058 A1, published Aug. 15, 2019. According to some examples, the nutritional service 132 may provide recommendations based on the microbiome fingerprint, dietary fingerprint, microbiome ancestry data and/or other data.

The nutritional environment 106 may include a collection of computing resources (e.g., computing devices such as servers). The computing resources may include a number of computing, networking and storage devices in communication with one another. In some examples, the computing resources may correspond to physical computing devices and/or virtual computing devices implemented by one or more physical computing devices.

It should be appreciated that the nutritional environment 106 may be implemented using fewer or more components than are illustrated in FIG. 1. For example, all or a portion of the components illustrated in the nutritional environment 106 may be provided by a service provider network (not shown). In addition, the nutritional environment 106 could include various Web services and/or peer to peer network configurations. Thus, the depiction of the nutritional environment 106 in FIG. 1 should be taken as illustrative and not limiting to the present disclosure.

The data ingestion service 110 facilitates submission of data utilized by the microbiome service 120 and, in some configurations, the nutritional service 132. Accordingly, utilizing a computing device 102, an electronic collection device, an at home biological collection device or via in clinic biological collection, an individual may submit data 108 to the nutritional environment 106 via the data ingestion service 110. Some of the data 108 may be sample data, biomarker test data, and some of the data 108 may be non-biomarker test data such as photos, barcode scans, timing data, and the like.

A “biomarker” or biological marker generally refers to one or more measurable indicators (that may be combined using various techniques) of some biological state or condition associated with an individual. Stated another way, a biomarker may be anything that can be used as an indicator of particular disease, condition, health, state, or some other physiological state of an organism. A biomarker typically can be measured accurately (either objectively and/or subjectively) and the measurement is reproducible. By way of example, the following are considered biomarkers: blood glucose, triglycerides (TG), insulin, c-peptides, ketone body ratios, IL-6 inflammation markers, the expression of any specified gene or protein, hunger, fullness, body mass index (BMI), composition of a microbiome (including not only what strains are present, but the relative abundance of two or more strains in a microbiome), and the like. In practice, a good biomarker is often a combination of two or more measurable indicators combined in a simple or complex way; in some cases, the combination of more than one measurable indicator makes the biomarker more closely linked to the disease, condition, health, state, or some other physiological state of an organism.

The measured biomarkers can include many different types of health data such as microbiome data which may be referred to herein as “microbiome data”, blood data, glucose data, lipid data, nutrition data, wearable data, genetic data, biometric data, questionnaire data, psychological data (e.g., hunger, sleep quality, mood, . . . ), objective health data (e.g., age, sex, height, weight, medical history, . . . ), as well as other types of data. Generally, “health data” refers to any psychological, subjective, and/or objective data that relates to and is associated with one or more individuals. The health data might be obtained through testing, self-reporting, and the like. Some biomarkers change in response to eating food, such as blood glucose, insulin, c-peptides, and triglycerides and their lipoprotein components.

To understand the differences in nutritional responses for different users, dynamic changes in biomarkers caused by eating food such as a standardized meal (“postprandial responses”) can be measured. By understanding an individual's nutritional responses, in terms of blood biomarkers such as glucose, insulin, and triglyceride levels, or non-blood biomarkers such as the microbiome, a nutritional service may be able to choose or recommend food(s) that is/are more suited for that particular person.

Data may also be obtained by the data ingestion service 110 from other data sources, such as data source(s) 150. For example, the data source(s) 150 can include, but are not limited to microbiome data associated with one or more users, nutritional data (e.g., nutrition of particular foods, nutrition associated with the individual, and the like), health data records associated with the individual and/or other individuals, and the like.

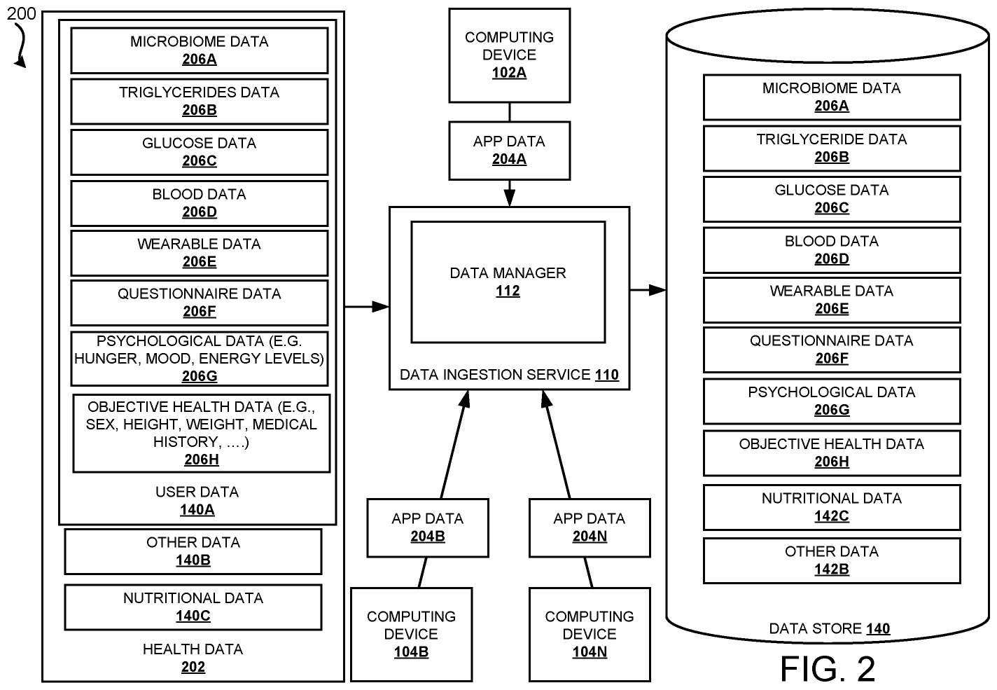

The data, such as data 108, or data obtained from one or more data sources 150, may then be processed by the data manager 112 and/or the microbiome manager 122 and included in a memory, such as the data store 140. As illustrated, the data store 140 can be configured to store user microbiome data 140A, other users' microbiome data 140A2, and other data 140B (see FIG. 2 for more details on the data ingestion service 110). In some examples, the user microbiome data 140A and other users' microbiome data 140A2 includes microbiome data.

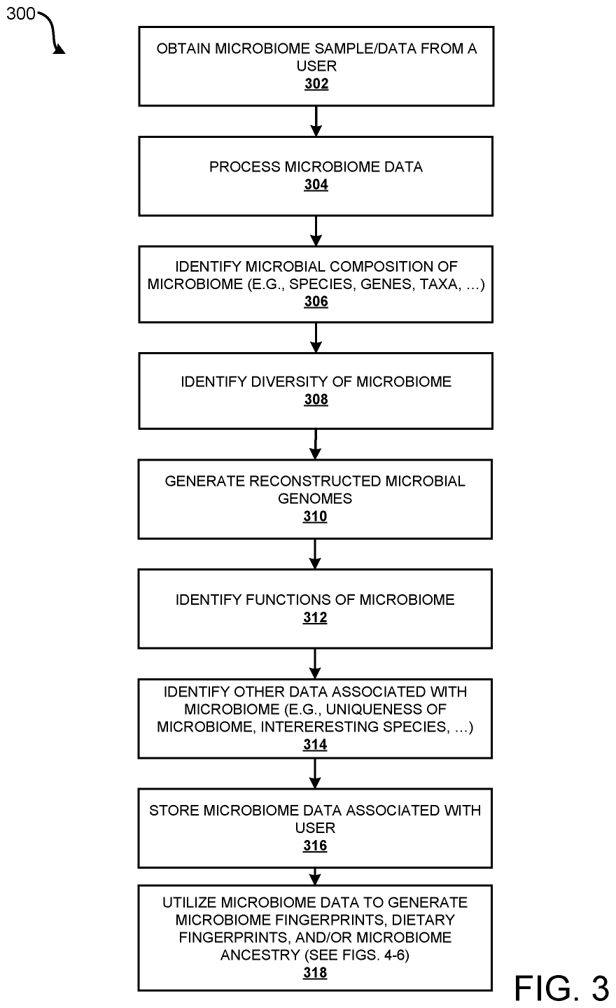

As discussed in more detail below (see FIGS. 3-7 for more details), the microbiome service 120 utilizing the microbiome manager 122, the microbiome analyzer 124, the microbiome finger printer 126, the microbiome dietary finger printer 128, and the microbiome ancestry manager 130, analyzes the data 108 associated with a user and generate a microbiome fingerprint, a dietary fingerprint, and microbiome ancestry data for the user. According to some configurations, the microbiome service 120 utilizes both data 108 associated with the user and data from other users.

In some examples, the microbiome manager 122 may utilize one or more machine learning mechanisms. For example, the microbiome manager 122 can use a classifier to classify the microbiome within a classification category (e.g., associate with a particular dietary index, a geographic location, . . . ). In other examples, the microbiome manager 122 may use a scorer to generate scores that may provide an indication of the dietary index associated with a user, how closely related the user is to other users based on the microbiome data, and the like.

The data ingestion service 110 and/or the microbiome service 120 can generate one or more user interfaces, such as a user interface 104 and/or user interface 104B, through which an individual, utilizing the computing device 102, or some other computing device, may provide/receive data from the nutritional environment 106. For example, the data ingestion service 110 may provide a user interface 104 that allows an individual of the computing device 102A to submit data 108 to the nutritional environment 106.

In some cases, the individual can also provide biological samples to a lab for testing, for instance using a biological collection device. According to some configurations, this will include At Home Blood Tests. According to some configurations, individuals can provide a sample (such as a stool sample) for microbiome analysis. As an example, metagenomic testing can be performed using the sample to allow the DNA of the microbes in the microbiome of an individual to be digitalized. Generally, a microbiome analysis includes determining the composition and functional potential (here called just “function”) of a community of microbes in a particular location, such as within the gut of an individual. An individual's microbiome appears to have a strong relationship to metabolism, weight, and health, yet only ten to thirty percent of the bacterial species in a microbiome is estimated to be common across different individuals. Embodiments described herein combine different techniques to assist in improving the accuracy of the data captured outside of a clinical setting, such as calculating accurate glucose responses to individual meals, which can then be linked to measures like the microbiome.

According to some configurations, individuals can provide a sample or samples of their stool for microbiome analysis as part of the at home biological collection. In some cases, this sample may be collected without using a chemical buffer. The sample can then be used to culture live microbes, or for chemical analysis such as for metabolites or for genetic related analysis such as metagenomic or metatranscriptomic sequencing. In such cases, the sample may suffer from changes in microbial composition due to causes including microbial blooming from oxygen in the period between being collected and when it is received in the lab, where it usually will be immediately assayed or frozen. In some cases, to avoid this change in bacterial composition after collection, the sample obtained a home may be frozen at low temperatures very rapidly after collection. The sample can then be used to culture live bacteria, or for chemical analysis or for metagenomic sequencing. This collection can be done as part of an in clinic biological collection or at home where the collection kit is configured to deliver such low temperatures and maintain them until a courier has taken the sample to a lab.

A stool sample may be combined with a chemical preservation buffer, such as ethanol, as part of the at home collection process to stop further microbial activity, which allows a sample to be kept at room temperature before being received at the lab where the assay is done. In some examples, the buffer may be a proprietary chemical product sold and validated by another company for the task of freezing microbial activity while still allowing the sample to be processed for metagenomics sequencing. A buffer allows for such a sample to be posted in the mail without (or minimizing) issues of microbial blooming or other continuing changes in microbial composition. The buffer may however prevent some biochemical analyses from being done, and because preservation buffers are likely to kill a large fraction of the microbial population, it is unlikely that samples conserved in preservation buffers can be used for cultivation assays.

In some cases, a user may do multiple stool tests over time, so that changes in the microbiome over time can be measured, or changes in the microbiome in response to meals, or changes in the microbiome in response to other clinical or lifestyle variations.

In some examples, the stool sample is collected using a scoop or swab from a stool that is collected by the user using a stool collection kit that prevents the stool from contamination, such as for instance the contamination that would occur from stool falling into a toilet. Because there is a very high microbial load in the gut microbiome compared, for example, to the skin microbiome, it is also possible that in some cases the stool sample is taken from paper that is used to clean the user's behind after they have passed a stool. This is only possible if the quantity of stool is large enough that the microbes from the stool greatly exceed the microbes that will be picked up from the user's skin or environmental contaminants. In any of these cases the scoop, swab, or tissue may be placed inside a collection device, such as a vial that contains a buffer solution. If the user ensures the stool comes into contact with the buffer, for example by shaking, then further microbial activity is stopped and the solution can be kept at room temperature without a significant change in microbial composition.

In some cases, a sterile synthetic tissue is used that does not have biological origins such as paper, so that when the DNA of the sample is extracted there is no contamination from DNA originating in the tissue.

According to some examples, the tissue is impregnated with a liquid to help capture more stool from the user's skin, where the liquid does not interfere with the results of the stool test and is not potentially dangerous for the human body.

In some cases, the timing and quality of the stool sample can be recorded using the computing device 102, for example using a camera. Where there are multiple stool tests the computing device 102 can use a barcode (or some other identifier) to confirm the timing and identity of that particular sample. Other data can also be collected. For example, data about how the sample was stored, how long the sample was stored before being supplied to the lab for analysis, and the like.

While the data ingestion service 110, the microbiome service 120, the nutritional service 132 are illustrated separately, all or a portion of these services may be located in other locations or together with other components. For example, the data ingestion service 110 may be located within the microbiome service 120. Similarly, the microbiome manager 122 may be part of a different service, and the like.

According to some examples, some individuals may be asked to visit a clinic to combine at home data with data collected at a clinic. The purpose of the clinic visit is to allow much higher accuracy of measurement for a subset of the individual's data, which can then be combined with the lower quality at home data. This may be used by the microbiome service 120 to improve the quality of the at home data.

According to some examples, the day before the visit to the clinic, the individuals are asked to avoid taking part in any strenuous exercise and to limit the intake of alcohol. In some configurations, the microbiome service 120 can analyze the data 108, such as data obtained from an activity tracker, to determine whether the individual followed the instructions of avoiding strenuous exercise. Similarly, the nutritional service 132, or some other device or component, may analyze the foods eaten by the individual by analyzing food data that indicates the foods eaten by the user. Individuals may be provided with instructions for the tests (e.g., avoid eating high fat or high fiber meals that may interfere with test results, fasting, drinking water, . . . ).

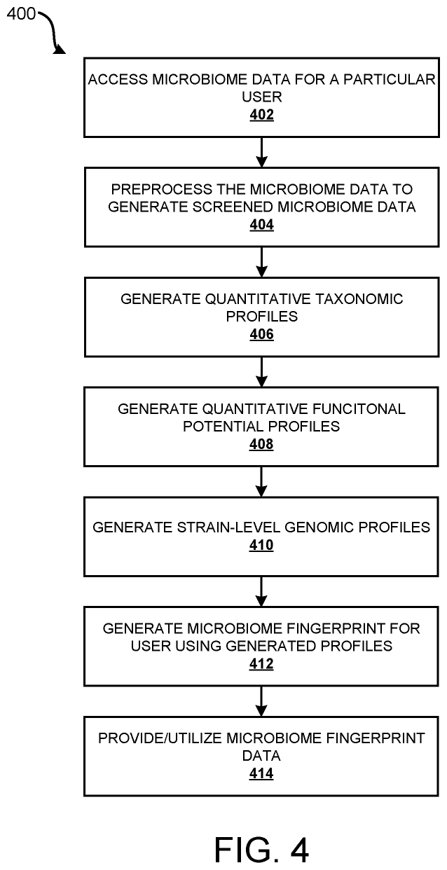

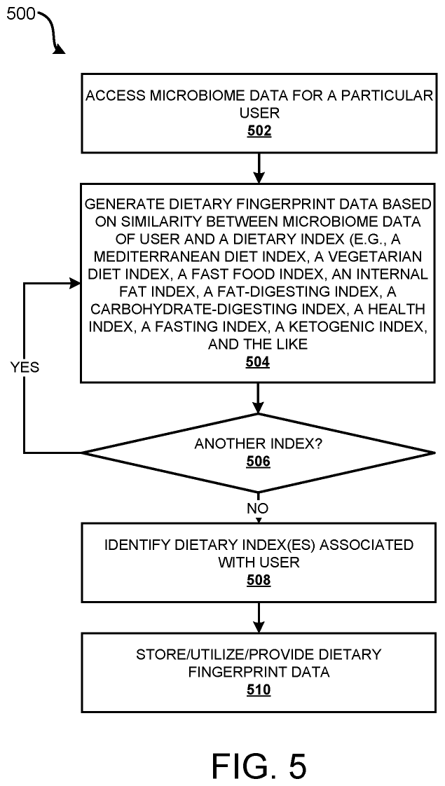

As described in more detail below with regard to FIGS. 4 and 5, the microbiome service 120 may use the microbiome manager 122 to generate a microbiome fingerprint, and a dietary fingerprint for a user. As discussed above, a “microbiome fingerprint” is data that uniquely identifies the microbiome of a user at a particular point in time. According to some configurations, the microbiome finger printer 126 generates a microbiome fingerprint from a user based on different profiles generated from the microbiome data, such as but not limited to quantitative taxonomic profiles, quantitative functional potential profiles, and strain-level genomic profiles. In some examples, the profiles are generated by the microbiome finger printer 126 and/or the microbiome analyzer 124.

According to some configurations, the microbiome fingerprint is a combination of descriptors, including, but not limited to (1) the quantitative (i.e. relative abundance) taxonomic profiles (i.e., the names or more generally identifiers (IDs) in case of unknown entities of microbial species or other taxonomic units), (2) the quantitative (i.e. relative abundance) functional potential profiles, (i.e., the names or generally identifiers (IDs) in case of unknown entities of microbial gene families, microbial pathways, and microbial functional modules), and (3) the strain-level genomic profiles (i.e., the reconstruction of the genomes or part of the genomes of as many microbes present in the microbiome as possible).

The microbiome fingerprint may be generated by the microbiome finger printer 126 using various techniques and methods. In some configurations, generation of the microbiome fingerprint includes obtaining the microbiome sample, generating DNA from the sample, preprocessing the raw sequencing data to the generate quality-screened sequencing data, and transforming the sequencing data is transformed into the numerical and genomics sets for the descriptors utilized to generate the microbiome fingerprint (e.g., quantitative taxonomic profiles, quantitative functional potential profiles, and strain-level genomic profiles).

The microbiome analyzer 124 may also be configured to perform processing associated with the microbiome data. For example, the microbiome analyzer 124 may be configured to generate and/or process sequencing data associated with the microbiome of the user. See FIG. 4 for more details on generating the profiles. After generating the profiles, the microbiome finger printer 126 may generate the microbiome fingerprint for the user. In some examples, the dietary finger printer 128 combines the data associated with the different profiles generated.

The dietary finger printer 128 is configured to generate a dietary fingerprint for the user. As discussed above, the “dietary fingerprint” of a user indicates how the microbiome of a user is associated with one or more different indexes that may be associated with a particular diet and/or a health characteristic. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like.

According to some configurations, the dietary finger printer 128 generates a score for each of the different indexes, such as from 0-100 (or some other indicator), to indicate how closely the microbiome of the user is associated with a particular index. For example, the dietary finger printer 128 may generate a score for each of the indexes based on how closely the microbiome of the user resembles a typical microbiome of someone that is known to follow a specific diet. For example, a score of 100 may indicate that the diet is strongly correlated to a particular diet, a score of 0 would indicate no correlation, and a score between 0 and 100 would indicate a different correlation. According to some configurations, the dietary finger printer 128 generates a Mediterranean diet index score, a vegetarian diet index score, a fast food index score, an internal fat index score, a fat-digesting index score, a carbohydrate-digesting index score, a health index score, fasting index score, ketogenic index score, and the like.

The Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

In other configurations, the dietary finger printer 128, or some other service or component may utilize different mechanisms to determine whether the microbiome of the user resembles a particular diet and/or group. For instance, the dietary finger printer 128 may utilize a machine learning mechanism to classify the microbiome of the user within a classification and/or generate a score, or some other indicator that indicates how closely the microbiome data of the user matches the microbiome data of a representative user associated with the particular index.

The microbiome ancestry manager 130 is configured to generate microbiome ancestry data for a user. A “microbiome ancestry” refers to microbiome data that indicates that the user has relationships with other users and/or locations. In some examples, the microbiome service analyzes the microbiome data of the user and determines how closely the microbiome of the user is related to other users, and/or locations. For instance, the microbiome service may determine a number of other users to which the microbiome of the user is most closely related to. In some configurations, the microbiome ancestry manager 130 compares the microbiome data of the user to microbiome data of other users to identify a relationship. Similar to generating the scores for the different indexes performed by the dietary finger printer 128, the microbiome ancestry manager 130 may generate a score for each comparison between the user and the other users. The scores that indicate a close relationship (e.g., above a specified value) with the user may be identified as related.

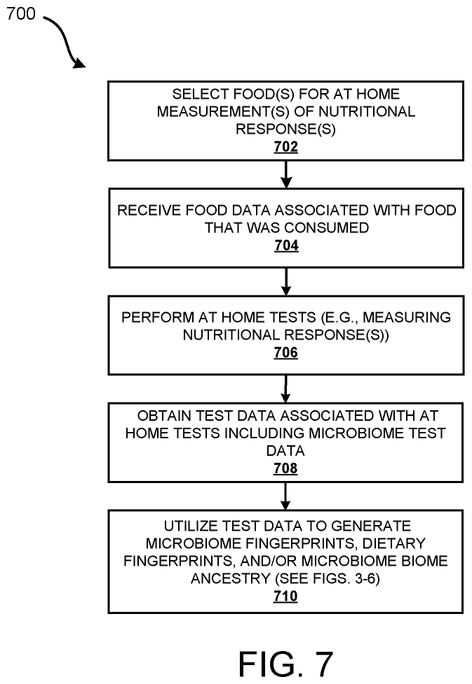

The microbiome service may also identify one or more locations to which the microbiome of the user is associated with. For example, the microbiome service may identify the countries the microbiome of the user is associated with (e.g. 75% North America, 25% Mexico). This identification may be based on microbiome data of users at different locations and/or different populations (e.g., English, American, French, Mexican, Italian, . . . ). See FIG. 7 for additional details for generating the microbiome ancestry data.

The microbiome analyzer 124, or some other device or component, may analyze the microbiome data of a user before/after generating the microbiome fingerprint, dietary fingerprint, and/or microbiome ancestry for a user. For example, the microbiome analyzer 124 may perform an analysis of the microbiome data to identify the microbial composition of the microbiome (e.g., the species, genes, taxa, and the like). The microbiome service may also generate reconstructed microbial genomes, determine a diversity of the microbiome, identify functions of the microbiome, identify a uniqueness of the microbiome, identify interesting species, and the like.

In some examples, the microbiome data of the user is compared (e.g., by the microbiome service 120) with other data that is gathered about the user, as well as other users. For instance, users may provide responses to questionnaires, data about food that is eaten, sleep habits, and the like. Among other uses, this data may be utilized to determine a “microbiome ancestry” of a user.

In some examples, the microbiome service may provide a user interface (UI), such as a graphical user interface (GUI) 104 for a user to view and interact with data associated with the microbiome fingerprints, dietary fingerprints, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, recommendation data that identifies one or more recommendations relating to changing the microbiome of the user, and the like. In some configurations, the user may utilize an application 130 on the computing device 102 to interact with the nutritional environment. In some configurations, the application 130 may include functionality relating to processing at least a portion of the data 108.

As an example, the microbiome service 120 may provide recommendations generated by the nutritional service 132 to increase the diversity of foods eaten as there is no one good food for a microbiome. The recommendations may include to eat different gut-healthy foods, eat fermented foods, minimize highly processed foods (things like emulsifiers and artificial sweeteners may affect the microbiome). The microbiome service may base the recommendations on data obtained from the user, and other users.

The microbiome service 120 may also track the state of the microbiome of the user over time. For example, the microbiome service may provide data related to different microbiome analysis. In this way, the user may see how changes made by the user (e.g., eating different foods, changing exercise patterns, . . . ) have affected the microbiome.

FIG. 2 is a block diagram depicting an illustrative operating environment 200 in which a data ingestion service 110 receives and processes data associated with data associated with at home tests and sample collections. As illustrated in FIG. 2, the operating environment 200 includes the data ingestion service 110 that may be utilized in ingesting data utilized by the microbiome service 120.

In some configurations, the data manager 112 is configured to receive data such as, health data 202 that can include, but is not limited to microbiome data 206A, triglycerides data 206B, glucose data 206C, blood data 206D, wearable data 206E, questionnaire data 206F, psychological data (e.g., hunger, sleep quality, mood, . . . ) 206G, objective health data (e.g., height, weight, medical history, . . . ) 206H, nutritional data 140B, and other data 140C.

According to some examples, the microbiome data 206A includes data about the gut microbiome of an individual. The gut microbiome can host a large number of microbial species (e.g., >1000) that together have millions of genes. Microbial species include bacteria, fungi, parasites, viruses, and archaea. Imbalance of the normal gut microbiome has been linked with gastrointestinal conditions such as inflammatory bowel disease (IBD) and irritable bowel syndrome (IBS), and wider systemic manifestations of disease such as obesity and type 2 diabetes (T2D). The microbes of the gut undertake a variety of metabolic functions and are able to produce a variety of vitamins, synthesize essential and nonessential amino acids, and provide other functions. Amongst other functions, the microbiome of an individual provides biochemical pathways for the metabolism of non-digestible carbohydrates; some oligosaccharides that escape digestion; unabsorbed sugars and alcohols from the diet; and host-derived mucins.

The triglycerides data 206B may include data about triglycerides for an individual. In some examples, the triglycerides data 206B can be determined from an At Home Blood Test which in some cases is a finger prick on to a dried blood spot card.

The glucose data 206C includes data about blood glucose. The glucose data 206C may be determined from various testing mechanisms, including at home measurements, such as a continuous glucose meter.

The blood data 206D may include blood tests relating to a variety of different biomarkers. As discussed above, at least some blood tests can be performed at home. In some configurations, the blood data 206D is associated with measuring blood sugar, insulin, c-peptides, triglycerides, IL-6 inflammation, ketone bodies, nutrient levels, allergy sensitivities, iron levels, blood count levels, HbA1c, and the like.

The wearable data 206E can include any data received from a computing device associated with an individual. For instance, an individual may wear an electronic data collection device 103, such as an activity-monitoring device, that monitors motion, heart rate, determines how much an individual has slept, the number of calories burned, activities performed, blood pressure, body temperature, and the like. The individual may also wear a continuous glucose meter that monitors blood glucose levels.

The questionnaire data 206F can include data received from one or more questionnaires, and/or surveys received from one or more individuals. The psychological data 206G, that may be subjectively obtained, may include data received from the individual and/or a computing device that generates data or input based on a subjective determination (e.g., the individual states that they are still hungry after a meal, or a device estimates sleep quality based on the movement of the user at night perhaps combined with heart rate data). The objective health data 206H includes data that can be objectively measured, such as but not limited to height, weight, medical history, and the like.

The nutritional data 140B can include data about food, which is referred to herein as “food data”. For example, the nutritional data can include nutritional information about different food(s) such as their macronutrients and micronutrients or the bioavailability of its nutrients under different conditions (raw vs cooked, or whole vs ground up). In some examples, the nutritional data 140C can include data about a particular food. For instance, before an individual consumes a particular meal, information about that food can be determined. As briefly discussed, the user might scan a barcode on the food item(s) being consumed and/or take one or more pictures of the food to determine the food, as well as the amount of food, being consumed.

The nutritional data can include food data that identifies foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes or rice), since carbohydrates may be altered by processes such as starch retrogradation. The food state can also be relevant for quantity estimation of the foods, since foods can change weight dramatically during cooking. In some instances, the user may also take a picture before and/or after consuming a meal to determine what food was consumed as well as how much of the food was consumed. The picture can also provide an indication as to the food state.

The other data 142B can include other data associated with the individual. For example, the other data 142B can include data that can be received directly from a computer application that logs information for an individual (e.g., food eaten, sleep, . . . ) and/or from the user via a user interface.

In some examples, different computing devices 102 associated with different users provide application data 204 to the data manager 112 for ingestion by the data ingestion service 110. As illustrated, computing device 102A provides app data 204A to the data manager 112, computing device 104B provides app data 204B to the data manager 112, and computing device 104N provides app data 204N to the data manager 112. There may be any number of computing devices utilized.

As discussed briefly above, the data manager 112 receives data from different data sources, processes the data when needed (e.g., cleans up the data for storage in a uniform manner), and stores the data within one or more data stores, such as the data store 140.

The data manager 112 can be configured to perform processing on the data before storing the data in the data store 140. For example, the data manager 112 may receive data for ketone bodies and then use that data to generate ketone body ratios. Similarly, the data manager 112 may process food eaten and generate meal calories, number of carbohydrates, fat to carbohydrate rations, how much fiber consumed during a time period, and the like. The data stored in the data store 140, or some other location, can be utilized by the microbiome service 120 to determine an accuracy of at home measurements of nutritional responses performed by users. The data outputted by the microbiome service 120 to the nutritional service may therefore contain different values than are stored in the data store 140, for example if a food quantity is adjusted.

FIGS. 3-7 are flow diagrams showing processes 300, 400, 500, 600, and 700, respectively that illustrate aspects of generating microbiome fingerprints, dietary fingerprints, and microbiome ancestry data in accordance with examples described herein. It should be appreciated that at least some of the logical operations described herein with respect to FIGS. 3-7, and the other FIGS., may be implemented (1) as a sequence of computer implemented acts or program modules running on a computing system and/or (2) as interconnected machine logic circuits or circuit modules within the computing system.

The implementation of the various components described herein is a matter of choice dependent on the performance and other requirements of the computing system. Accordingly, the logical operations described herein are referred to variously as operations, structural devices, acts, or modules. These operations, structural devices, acts, and modules may be implemented in software, in firmware, in special purpose digital logic and any combination thereof. It should also be appreciated that more or fewer operations may be performed than shown in the FIGS. and described herein. These operations may also be performed in parallel, or in a different order than those described herein.

FIG. 3 is a flow diagram showing a process 300 illustrating aspects of a mechanism disclosed herein for obtaining and utilizing microbiome data for a user to generate microbiome fingerprints, dietary fingerprints, and microbiome ancestry for users.

The process 300 may begin at 302, where microbiome sample/data is obtained from a user. As discussed above, a user may provide one or more microbiome samples that may be obtained at home or in a clinical setting. For example, the user may provide a sample or samples of their stool for microbiome analysis as part of the at home biological collection, and/or the sample(s) may be collected in a lab, or other clinical setting. In some configurations, the user may also provide other data that may be utilized when processing the sample. For instance, the user may provide timing data indicating when the sample was taken, conditions under which the sample was obtained, and/or other health data.

At 304, the microbiome data is processed. As discussed above, microbiome service 120 may generate DNA data from the sample. In some examples, the DNA is extracted from the cells of the microbiome sample and purified. Different techniques that are commercially available can be utilized for DNA extraction from the microbiome sample. Generally, the use of different extraction techniques may result in different biases that may affect an accurate microbial representation.

At 306, the microbial composition of the microbiome sample may be identified. According to some configurations, the microbiome service 120, or some other device or component, identifies the microbial composition of the microbiome (e.g., the species, genes, taxa, and the like). The microbiome service 120 may also generate reconstructed microbial genomes, determine a diversity of the microbiome, identify functions of the microbiome, identify a uniqueness of the microbiome, identify interesting species, and the like.

At 308, the diversity of the microbiome may be determined. As discussed above, the microbiome service 120 may determine the diversity of the microbiome associated with a user. In some examples, the diversity determined by the microbiome service 120 is the number of individual bacteria from each of the bacterial species present in the microbiome. Having a more diverse microbiome may have health benefits. According to some configurations, the microbiome service 120 may provide this data, possibly along with recommendations, to the user via a UI, or some other interface.

At 310, reconstructed microbial genomes are generated. The microbiome service 120, or some other component or device may generate the reconstructed microbial genomes. Reconstruction of DNA fragments into genomes may utilize different techniques and methods and generally incorporates sequence assembly and sorting/clustering of assembled sequences into different bins associated with characteristic of a genome.

At 312, the functions of a microbiome may be determined. As discussed above, the microbiome service 120, or some other device or component, may determine the functions of a microbiome. Different techniques and methods may be utilized to determine the functions. Generally, the microbiome service 120 may map the sequencing reads against sequences of DNA (or amino acids) representing known genes (or proteins) and gene families (or protein families) to determine the functional potential of the microbiome.

At 314, other data associated with the microbiome of the user may be determined. As discussed above, the microbiome service 120, or some other device or component, may determine data such as the uniqueness of the microbiome (e.g., compared to the microbiome of other users), species identified as interesting, and the like.

At 316, the microbiome data associated with the user is stored. As discussed above, the microbiome service 120, or some other device or component, may store the microbiome data in a data store, such as user microbiome data 140A within data store 140.

At 318, the microbiome data associated with the user is utilized to generate microbiome fingerprints, dietary fingerprints, and microbiome ancestry for the user. As discussed above, the microbiome service 120, or some other device or component, may perform these tasks. See FIGS. 4-6 and related discussion for more details.

FIG. 4 is a flow diagram showing a process 400 illustrating aspects of a mechanism disclosed herein for generating a microbiome fingerprint for a user. As discussed above, the microbiome fingerprint may be generated using various techniques and methods. The following process is an example of generating a microbiome fingerprint.

At 402, microbiome data for a particular user is accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 404, the microbiome data may be preprocessed to generate screened microbiome data. As discussed above, the microbiome service 120, or some other device or component, may process the sequencing data to generate screened sequencing data. The screened sequence data may make the generation of the different profiles described below be more accurate.

At 406, the quantitative taxonomic profiles are generated. As discussed above, the microbiome service 120, or some other device or component, may generate the quantitative taxonomic profiles. The quantitative taxonomic profiles can be obtained by mapping (i.e. matching the sequences) the sequencing reads against sequences representing the known microbial organisms. The mapping is then processed to produce relative abundances of the reference microbes. Many open source algorithms and corresponding implementations are available for this step, including for example, the techniques as described by Truong et al. (Nature Methods 12 (10): 902-3, 2015) and the newer versions of the associated software.

At 408, the quantitative functional potential profiles are generated. As discussed above, the microbiome service 120, or some other device or component, may generate the quantitative functional potential profiles. The quantitative functional potential profiles can be obtained by mapping the sequencing reads against sequences of DNA (or amino acids) representing known genes (or proteins) and gene families (or protein families). Based on the number of reads matching each gene or gene family the presence and abundance of the gene families and pathways are inferred. Several open source algorithms and corresponding implementations are available for this step, including for example the technique HUMAnN2 as described by Abubucker et al. (PLOS Computational Biology 8 (6), 2012) and Franzosa et al. (Nature Methods, 15(11), 962, 2018) and any newer versions of the associated software.

At 410, the strain-level genomic profiles are generated. As discussed above, the microbiome service 120, or some other device or component, may generate the strain-level genomic profiles. The strain-level genomic profiles, or the third descriptor, can be obtained with reference-based and assembly-based approaches. For reference-based approaches the methods use specific genetic markers against which the reads are mapped, and single-nucleotide polymorphisms are inferred. The combinations of single-nucleotide polymorphisms provide strain-specific profiles. Some open source algorithms and implementations for this step are available, including for example the techniques described by Truong et al. (Genome Research 27 (4): 626-38, 2017). In assembly-based approaches, reads may be first concatenated to form longer contiguous sequences such as described by Li et al. (Bioinformatics 31 (10): 1674-76, 2015).

Contigs may then be clustered in bins representing the sequences of whole genomes, such as described by Kang et al. (PeerJ 7: e7359, 2019). The resulting draft genomes may be quality controlled using for example the techniques described by Parks et al. (Genome Research 25 (7): 1043-55, 2015). The quality-controlled genomes represent single strains in the microbiome.

At 412, the microbiome fingerprint for the user is generated. As discussed above, the microbiome service 120, or some other device or component, may combine the data associated with the different indexes generated at 406, 408, and 410 to generate the microbiome fingerprint for the user.

FIG. 5 is a flow diagram showing a process 500 illustrating aspects of a mechanism disclosed herein for generating a dietary fingerprint for a user.

The process 500 may begin at 502, where microbiome data for a particular user are accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 504, dietary fingerprint data is generated. As discussed above, the microbiome service 120, or some other device or component, may generate dietary fingerprint data that identifies a similarity between the microbiome of a particular user and a “dietary fingerprint” is data that identifies how the microbiome of a user is associated with one or more different indexes. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like. According to some configurations, one or more computers of a microbiome service generate a score, such as from 0-100, (or some other indicator) that indicates how closely the microbiome of the user is associated with a particular index.

As an example, the Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

At 506, a determination is made as to whether another dietary index is to be compared. As discussed above, there may be a variety of dietary indexes, including but not limited to a Mediterranean diet index, a vegetarian diet index, a fast food index, an internal fat index, a fat-digesting index, a carbohydrate-digesting index, a health index, a fasting index, a ketogenic index, and the like. When there is another index, the process 500 returns to 504. When there is not another index, the process 500 moves to 508.

At 508, the dietary index(es) associated with the user are identified. As discussed above, the microbiome service 120, or some other device or component, may identify one or more diets that resemble the microbiome of the user. In some examples, the microbiome service 120 identifies the closest dietary index (e.g., based on a score). In other examples, the microbiome service 120 may rank the dietary index.

At 510, the dietary fingerprint data may be utilized. As discussed above, the microbiome service 120, or some other device or component, may utilize the dietary fingerprint data when providing data to the user, when generating the microbiome ancestry data, generating recommendations for the user (e.g., nutritional), and/or performing some other task.

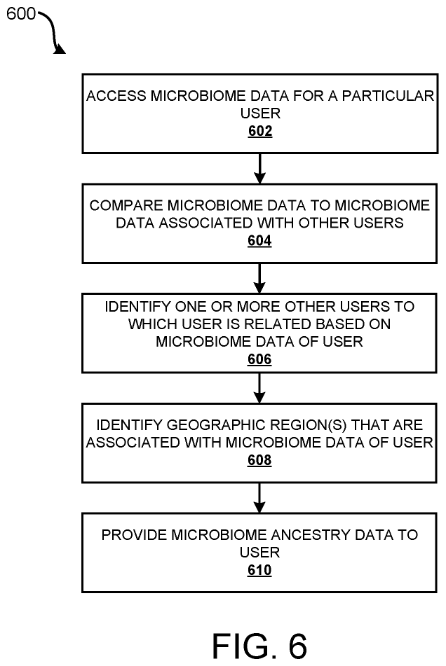

FIG. 6 is a flow diagram showing a process 600 illustrating aspects of a mechanism disclosed herein for generating a microbiome ancestry for a user.

The process 600 may begin at 602, where microbiome data for a particular user is accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 604, the microbiome data is compared to microbiome data from other users. As discussed above, the microbiome service 120, or some other device or component, may utilize the microbiome data, such as the microbiome fingerprint data of a particular user, and compare microbiome fingerprint data of other users. According to some configurations, the microbiome service 120 may generate one or more indicators that identify how close another user is to the user based on a similarity of the microbiome data.

At 606, one or more other users are identified based on a similarity of the microbiome data between the users. As discussed above, the microbiome service 120, or some other device or component, may identify the related users based on a score generate the microbiome service 120, or some other indicators.

At 608, the geographic region(s) that are commonly associated with the microbiome data of a user are identified. As discussed above, the microbiome service 120, or some other device or component, may identify that different geographic regions are more closely linked to certain microbiomes.

At 610, the microbiome ancestry data may be utilized. As discussed above, the microbiome service 120, or some other device or component, may utilize the microbiome ancestry data when providing data to the user, when generating the microbiome ancestry data, generating recommendations for the user (e.g., nutritional), and/or performing some other task.

FIG. 7 is a flow diagram showing a process 700 illustrating aspects of a mechanism disclosed herein for obtaining test data, including microbiome data, to be utilized for generating microbiome fingerprints, dietary fingerprints, and microbiome ancestry for users.

At 702, food(s) for at home measurements of nutritional responses may be selected. As briefly discussed above, different foods may be selected for a user to eat before a test is performed in order to evoke a desired response. The foods can include foods for a series of standardized meals, a single food, or some other combination of foods.

At 704, food data is received. As discussed above, the food data is associated with foods that are utilized to evoke a nutritional response. The food data can include foods for a series of standardized meals, a single food, or some other combination of foods. The food data can include data such as foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes or rice), since carbohydrates may be altered by processes such as starch retrogradation. The food state can also be relevant for quantity estimation of the foods, since foods can change weight dramatically during cooking.

At 706, at home test(s) are performed. The tests may include at home tests as described above and/or the collection of one or more samples (e.g., stool for microbiome analysis).

At 708, test data associated with the at home tests including microbiome data is received. As discussed above, microbiome data may be associated with one or more tests. In some configurations, the microbiome data includes a stool sample, timing data for the sample (e.g., when collected, how long stored before providing to a lab), data associated with collection of the sample (e.g., how was sample stored, was the sample contaminated), as well as other data. For example, a user may be instructed to take a picture of the sample and provide the image to the service.

At 710, the test data is utilized to generate microbiome fingerprints, dietary fingerprints, and microbiome ancestry. In some examples, the test data is used by the microbiome service 120 to generate the microbiome fingerprints, dietary fingerprints, and microbiome ancestry. The nutritional service 132 may also use the test data to generate nutritional recommendations (for instance in the form of a diet program or food guidance program) that are personalized for a particular user.

### Detection and Identification of Individual Microbes.

One of ordinary skill in the art will recognize that myriad methods exist for detecting and identifying individual member microbes in the microbiome of a subject, as well as methods for identifying and quantifying (in relative or absolute terms) the members of a microbiome. See, for instance: Asnicar et al. (Nat Med. 27:321-323, 2021), Davidson & Epperson (Methods Mol. Biol., 1706:77-90, 2018), Nagpal et al. (Front Microbiol., 8:2897, doi: 10.3389/fmicb.2018.02897, 2018), Nagpal et al. (Sci Rep. 8(1): 12649, 2018), The Integrative HMP (iHMP) Research Network Consortium (Nature 569:641-648, 2019; and publications cited therein), Wu et al. (Gut. 65(1): 63-72, 2016). Additional resources are available online, for instance, through the NIH Human Microbiome Project (at hmpdacc.org), including tools and protocols related to Microbial Reference Genomes, Sampling, Sequence & Analysis of 16S RNA, and Sampling, Sequencing & Analysis of Whole Metagenomic Sequence. See also: WO 2021/165494 “Generating Microbiome Fingerprints, Dietary Fingerprints, and Microbiome Ancestry”; and PCT/EP2021/057116 “Microbiome Fingerprints, Dietary Fingerprints, and Microbiome Ancestry, and Methods of Their Use”.

Representative specific individual microbes and sets of microbes associated with and/or linked to poor health and others associated with and/or linked to pro-health conditions are described herein. Microbial profiles that include these microbes or sets of microbes can be detected without needing to sequence or otherwise assay the entire microbiome of the subject. For instance, the following are pro-health linked/indicator microbes: Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and the following are poor health linked/indicator microbes: Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli. These strains can be further identified by their respective NCBI Taxonomy ID Number (see ncbi.nlm.nih.gov/taxonomy), as shown in Table 1. Additional specific taxonomic information can be found, for instance, using MetaPhIAn2 (Metagenomic Phylogenetic Analysis; version 2.9.21 and marker database release 2.9.4; Truong et al., Nat. Methods 12, 902-903, 2015).

TABLE 1
NCBI Taxonomy Identification Numbers for Select Indicator Microbes

| Indicator: Pro-Health | | Indicator: Poor-Health | |
|---|---|---|---|
| NCBI: txid | Species label | NCBI: txid | Species label |
| 537011 | Prevotella copri | 169435 | Anaerotruncus colihominis |
| 12967 | Blastocystis spp. | 53443 | Blautia hydrogenotrophica |
| 28025 | Bifidobacterium animalis | 40520 | Blautia obeum |
| 1262777 | Clostridium sp CAG 167 | 208479 | Clostridium bolteae |
| 39485 | Eubacterium eligens | 1263064 | Clostridium bolteae CAG 59 |
| 1263006 | Firmicutes bacterium CAG 170 | 1522 | Clostridium innocuum |
| 1262988 | Firmicutes bacterium CAG 95 | 1262824 | Clostridium sp CAG 58 |
| 729 | Haemophilus parainfluenzae | 29348 | Clostridium spiroforme |
| 1897011 | Oscillibacter sp 57 20 | 1512 | Clostridium symbiosum |
| 1855299 | Oscillibacter sp PC13 | 147207 | Collinsella intestinalis |
| 454155 | Paraprevotella xylaniphila | 84112 | Eggerthella lenta |
| 301301 | Roseburia hominis | 39496 | Eubacterium ventriosum |
| 1262942 | Roseburia sp CAG 182 | 292800 | Flavonifractor plautii |
| 43675 | Rothia mucilaginosa | 360807 | Roseburia inulinivorans |
| 1520815 | Ruminococcaceae bacterium D5 | 33038 | Ruminococcus gnavus |
| 39778 | Veillonella dispar | 1535 | Clostridium leptum |
| 1911679 | Veillonella infantium | 1550024 | Ruthenibacterium |
| | | | lactatiformans |
| 853 | Faecalibacterium prausnitzii | 562 | Escherichia coli |
| 1115758 | Romboutsia ilealis | 169435 | Anaerotruncus colihominis |
| 39777 | Veillonella atypica | 53443 | Blautia hydrogenotrophica |

A collection of two or more microbes described or illustrated herein as associated with a biological status or condition can be referred to as a microbial signature, or a microbiome fingerprint, or a microbial profile. For instance, any two, any three, any four, any five, any six, any seven, any eight, any nine, any 10, any 11, any 12, any 13, any 14, any 15, or more microbes listed in Table 1 may be included in a microbial signature for a biological status or condition. Such microbes may be selected from the Pro-Health or the Poor Health indicators, or some from both. All seventeen of the listed pro-health indicator microbes for instance may be included in a single microbial signature. Similarly, all fifteen poor health indicator microbes may be included in a single microbial signature. Additional microbes useful in the assembling of a microbial signature, or microbiome fingerprint, are provided for instance in Asnicar et al. (Nat Med. 27:321-323, 2021).

### Representative Computer Architecture.

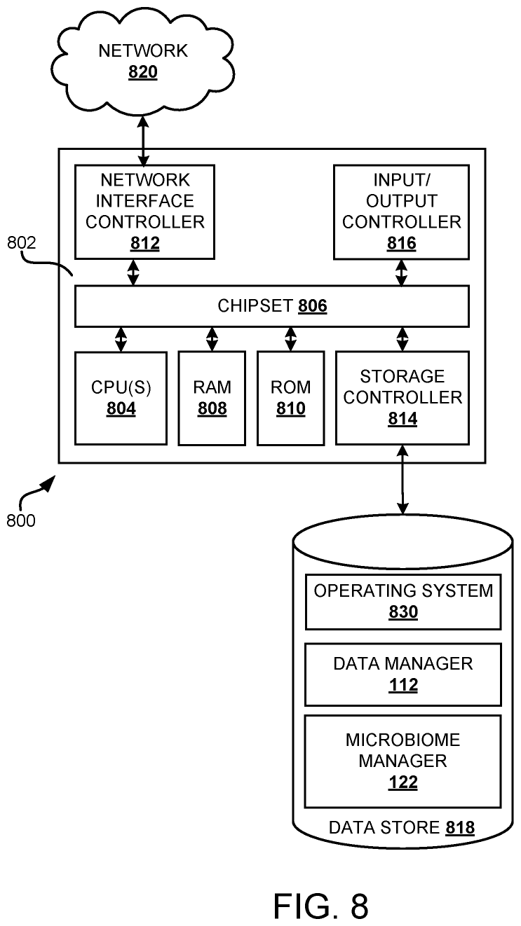

FIG. 8 shows an example computer architecture for a computer 800 capable of executing program components for generating microbiome fingerprints, dietary fingerprints, and microbiome ancestry for users in the manner described above. The computer architecture shown in FIG. 8 illustrates a conventional server computer, workstation, desktop computer, laptop, tablet, network appliance, digital cellular phone, smart watch, or other computing device, and may be utilized to execute any of the software components presented herein. For example, the computer architecture shown in FIG. 8 may be utilized to execute software components for performing operations as described above. The computer architecture shown in FIG. 8 might also be utilized to implement a computing device 102, or any other of the computing systems described herein.

The computer 800 includes a baseboard 802, or “motherboard,” which is a printed circuit board to which a multitude of components or devices may be connected by way of a system bus or other electrical communication paths. In one illustrative example, one or more central processing units (CPUs) 804 operate in conjunction with a chipset 806. The CPUs 804 may be standard programmable processors that perform arithmetic and logical operations necessary for the operation of the computer 800.

The CPUs 804 perform operations by transitioning from one discrete, physical state to the next through the manipulation of switching elements that differentiate between and change these states. Switching elements may generally include electronic circuits that maintain one of two binary states, such as flip-flops and electronic circuits that provide an output state based on the logical combination of the states of one or more other switching elements, such as logic gates. These basic switching elements may be combined to create more complex logic circuits, including registers, adders-subtractors, arithmetic logic units, floating-point units and the like.

The chipset 806 provides an interface between the CPUs 804 and the remainder of the components and devices on the baseboard 802. The chipset 806 may provide an interface to a random-access memory (RAM) 808, used as the main memory in the computer 800. The chipset 806 may further provide an interface to a computer-readable storage medium such as a read-only memory (ROM) 810 or non-volatile RAM (NVRAM) for storing basic routines that help to startup the computer 800 and to transfer information between the various components and devices. The ROM 810 or NVRAM may also store other software components necessary for the operation of the computer 800 in accordance with the examples described herein.

The computer 800 may operate in a networked environment using logical connections to remote computing devices and computer systems through a network, such as the network 820. The chipset 806 may include functionality for providing network connectivity through a network interface controller (NIC) 812, such as a mobile cellular network adapter, Wi-Fi network adapter or gigabit Ethernet adapter. The NIC 812 is capable of connecting the computer 800 to other computing devices over the network 820. It should be appreciated that multiple NICs 812 may be present in the computer 800, connecting the computer to other types of networks and remote computer systems.

The computer 800 may be connected to a mass storage device 818 that provides non-volatile storage for the computer. The mass storage device 818 may store system programs, application programs, other program modules and data, which have been described in greater detail herein. The mass storage device 818 may be connected to the computer 800 through a storage controller 814 connected to the chipset 806. The mass storage device 818 may include one or more physical storage units. The storage controller 814 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computer 800 may store data on the mass storage device 818 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 818 is characterized as primary or secondary storage and the like.

For example, the computer 800 may store information to the mass storage device 818 by issuing instructions through the storage controller 814 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 800 may further read information from the mass storage device 818 by detecting the physical states or characteristics of one or more particular locations within the physical storage units.

In addition to the mass storage device 818 described above, the computer 800 may have access to other computer-readable storage media to store and retrieve information, such as program modules, data structures, or other data. It should be appreciated by those skilled in the art that computer-readable storage media is any available media that provides for the non-transitory storage of data and that may be accessed by the computer 800.

By way of example, and not limitation, computer-readable storage media may include volatile and non-volatile, removable and non-removable media implemented in any method or technology. Computer-readable storage media includes, but is not limited to, RAM, ROM, erasable programmable ROM (EPROM), electrically-erasable programmable ROM (EEPROM), flash memory or other solid-state memory technology, compact disc ROM (CD-ROM), digital versatile disk (DVD), high definition DVD (HD-DVD), BLU-RAY, or other optical storage, magnetic cassettes, magnetic tape, magnetic disk storage or other magnetic storage devices, or any other medium that can be used to store the desired information in a non-transitory fashion.

The mass storage device 818 may store an operating system 830 utilized to control the operation of the computer 800. According to one example, the operating system includes the LINUX® (Linus Torvalds, Boston, MA) operating system. According to another example, the operating system includes the WINDOWS® SERVER® (Microsoft Corporation, Redmond, WA) operating system from MICROSOFT® (Microsoft Corporation, Seattle, WA). According to another example, the operating system includes the iOS® (Cisco Technology Inc., San Jose, CA) operating system from Apple® (Apple Inc., Cupertino, CA). According to another example, the operating system includes the Android® (Google LLC, Mountain View, CA) operating system from Google® (Google LLC) or its ecosystem partners. According to further examples, the operating system may include the UNIX® (The Open Group Limited, Reading, Berkshire, England) operating system. It should be appreciated that other operating systems may also be utilized. The mass storage device 818 may store other system or application programs and data utilized by the computer 800, such as components that include the data manager 122, the microbiome manager 122 and/or any of the other software components and data described above. The mass storage device 818 might also store other programs and data not specifically identified herein.

In one example, the mass storage device 818 or other computer-readable storage media is encoded with computer-executable instructions that, when loaded into the computer 800, create a special-purpose computer capable of implementing the examples described herein. These computer-executable instructions transform the computer 800 by specifying how the CPUs 804 transition between states, as described above. According to one example, the computer 800 has access to computer-readable storage media storing computer-executable instructions which, when executed by the computer 800, perform the various processes described above with regard to FIGS. 4-8. The computer 800 might also include computer-readable storage media for performing any of the other computer-implemented operations described herein.

The computer 800 may also include one or more input/output controllers 816 for receiving and processing input from a number of input devices, such as a keyboard, a mouse, a touchpad, a touch screen, an electronic stylus, or other type of input device. Similarly, the input/output controller 816 may provide output to a display, such as a computer monitor, a flat-panel display, a digital projector, a printer, a plotter, or other type of output device. It will be appreciated that the computer 800 may not include all of the components shown in FIG. 8, may include other components that are not explicitly shown in FIG. 8, or may utilize an architecture completely different than that shown in FIG. 8.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. A method, including: preparing a food guidance program for a subject; communicating the food guidance program to the subject; wherein, when the subject follows the food guidance program: presence or relative abundance of at least one pro-health indicator microbe in the microbiome of the subject is increased, wherein the one or more pro-health indicator microbes are selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; or presence or relative abundance of at least one poor health indicator microbe in the microbiome of the subject is decreased, wherein the one or more poor health indicator microbe selected from the group consisting of Clostridium sp CAG 58, Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Blautia hydrogenotrophica, Anaerotruncus colihominis, Ruminococcus gnavus, Flavonifractor plautii, Clostridium leptum, Ruthenibacterium lactatiformans, and Escherichia coli; or both.
2. Use of a diet program to improve the gut microbiome of a subject, wherein the improvement to the gut microbiome includes: increasing presence or relative abundance of at least one pro-health indicator microbe in the microbiome of the subject; decreasing presence or relative abundance of at least one poor health indicator microbe in the microbiome of the subject; or both.
3. The use of embodiment 2, wherein the diet program is prepared at least in part based on: presence, absence, or relative abundance of at least one pro-health indicator microbe in the microbiome of the subject; and/or presence, absence, or relative abundance of at least one poor health indicator microbe in the microbiome of the subject.
4. A method, including: preparing a food guidance program for a subject; communicating the food guidance program to the subject; wherein, when the subject follows the food guidance program: presence or relative abundance of at least one pro-health indicator microbe in the microbiome of the subject is increased; presence or relative abundance of at least one poor health indicator microbe in the microbiome of the subject is decreased; or both.
5. A method, including: detecting in a microbiome sample from a human subject one or more pro-health indicator microbes selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and modifying dietary intake of the human using a food guidance program that increases growth or survival of one or more of the pro-health indicator microbe(s); and/or detecting in a microbiome sample from the human subject one or more poor health indicator microbe selected from the group consisting of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and modifying dietary intake of the human using a food guidance program that decreases growth or survival of one or more of the poor health indicator microbe(s).
6. The method of embodiment 1, 4, or 5, or the use of embodiment 2 or 3, wherein the food guidance program is personalized for the subject.
7. A method of altering abundance of at least one microbial species in a gut microbiome of a subject, including: providing the subject with a personalized food guidance program, which personalized food guidance program is developed based at least in part on a food score personalized for the subject, and modifying food intake of the subject based on the personalized food guidance plan, thereby altering the abundance of at least one microbial species in the gut microbiome of the subject.
8. The method of embodiment 7, wherein altering abundance includes: increasing presence or relative abundance of at least one pro-health indicator microbe in the microbiome of the subject; decreasing presence or relative abundance of at least one poor health indicator microbe in the microbiome of the subject; or both.
9. A method of improving gut microbiome balance/health of a subject, including: developing a personalized food guidance program for the subject, communicating the personalized food guidance program to the subject, and modifying food intake of the subject, in accordance with the personalized food guidance program, thereby improving gut microbiome balance/health of the subject.
10. A method of improving a gut microbiome profile of a subject, including: assaying the gut microbiome of the subject, thereby producing a first microbiome signature of the subject; developing a personalized food guidance program for the subject, which personalized food guidance program will improve the gut microbiome of the subject when the subject follows the program; communicating the personalized food guidance program to the subject; assaying the gut microbiome of the subject at a time the after the subject begins to follow the customized diet guidance plan, thereby producing a second microbiome signature of the subject; comparing the first microbiome signature of the subject to the second microbiome signature of the subject, wherein the first and second microbiome signatures include the presence, absence, or relative abundance of one or more pro-health indicator microbes and/or the presence, absence, or relative abundance of one or more poor health indicator microbes; and wherein an increase in the presence or relative abundance of the one or more pro-health indicator microbes and/or a decrease in the presence or relative abundance of the one or more poor health indicator microbes from the first signature to the second signature constitutes an improved gut microbiome profile of the subject.
11. The use or method of embodiment 6, or the method of any of embodiments 7-10, wherein the personalized food guidance program is based at least based in part on one or more of: a non-microbial biomarker of the subject; a nutritional response of the subject; medical history of the subject; a health condition of the subject; predicted hunger of the subject; predicted response to food consumption of the subject; glucose response of the subject; fat response of the subject; microbiome data of the subject; data about the subject's overall health; and/or potential health risks for the subject.
12. The use or method of any one of embodiments 2-4, 8, 10, or 11, wherein: the pro-health indicator microbe is selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and/or the poor health indicator microbe is selected from the group including Clostridium sp CAG 58, Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Blautia hydrogenotrophica, Anaerotruncus colihominis, Ruminococcus gnavus, Flavonifractor plautii, Clostridium leptum, Ruthenibacterium lactatiformans, and Escherichia coli
13. The use or method of embodiment 1 or embodiment 12, wherein the pro-health indicator microbe includes Prevotella copri.
14. The use or method of embodiment 1, 12 or 13, wherein the poor health indicator microbe includes one or more of Clostridium sp CAG 58, Collinsella intestinalis, Blautia hydrogenotrophica, Flavonifractor plautii, Clostridium leptum, and/or Ruthenibacterium lactatiformans.
15. The use or the method of any one of embodiments 1-14, wherein the food guidance program is developed based at least in part based on or in reference to a database including one or more of: correlations between food consumed and microbiome members for thousands of individual subjects; and/or a plurality of foods designated as “to be promoted in a diet”, wherein this designation indicates the food tends to support a healthy condition in the subject, or tends to move a subject incorporating the food in their diet more toward a healthy state; and/or a plurality of foods designated as “to be demoted in a diet”, wherein this designation indicates the food tends to not support a healthy condition, or tends to support an unhealthy condition in the subject, or tends to move a subject incorporating the food in their diet more toward a less healthy state.
16. Use of a diet program, such as a personalized diet program, to improve the gut microbiome of a subject or to alter the altering abundance of at least one microbial species in a gut microbiome of a subject, essentially as disclosed herein.
17. Use of a diet program to improve the gut microbiome of a subject, where the improvement to the gut microbiome includes: increasing presence or relative abundance of at least one pro-health indicator microbe in the microbiome of the subject; decreasing presence or relative abundance of at least one poor health indicator microbe in the microbiome of the subject; or both.

### Example 1: Bad Bug Abundance Reduction after Personalized Diet Recommendations

This example describes an analysis of the impact of adherence to diet recommendations (exemplified with personalized diet recommendations) on the abundance of “good” gut microbiome microbes and “bad” gut microbiome microbes in human subjects. In this context, “good” microbes are positively (negatively) correlated with markers associated with good (poor) health, while “bad” microbes are negatively (positively) correlated with markers associated with poor (good) health. In this study, subjects were tested after 7-9 months of adherence to the diet recommendations.

### Gut Microbiome Profile

Sample collection and sequencing: At the beginning of the study, participants received a stool collection kit to collect a stool sample at home. The stool was deposited into a DNA/RNA Shield buffer tube for stability at ambient temperature and sent by standard mail to Zymo Laboratories.

Once at the laboratory, an unbiased DNA isolation was performed using ZymoBIOMICS-96 MagBead DNA Kit. The isolated microbial DNA is then prepared for shotgun metagenomics sequencing using the Nextera DNA Flex Library Prep Kit.

Each sample was sequenced with a depth of at least 3.75 Gb using the 150×2 chemistry on the NovaSeq platform (S4 flow cells).

The same procedure was repeated at the time of retesting, which was between 7 and 9 months after the initial test.

Metagenome quality control: All metagenomic sequence data underwent a quality control analysis as implemented in the pipeline available online at github.com/SegataLab/preprocessing. For each sample the pipeline (1) removed from low quality reads (quality score <Q20), removed too short reads (<75 bp), and reads with more than 2 ambiguous nucleotides; (2) screened and removed reads from the phi X 174 Illumina spike-in genome and human associated reads (hg19); (3) split and sorted the remaining cleaned reads in forward, reverse and unpaired read files.

Metagenome taxonomic profiling: The cleaned metagenome datasets were analysed by MetaPhIAn (Truong et al., Nat. Methods 12, 902-903, 2015) v.3.0 (Beghini et al., Elife. 10:e65088, 2021.doi: 10.7554/eLife.65088) to identify the sample taxonomic profiles and to quantify the microbe relative abundances. MetaPhIAn v.3.0 mapped the metagenomes against a marker gene database using Bowtie 2 with the parameter ‘very-sensitive’. The marker gene database consists of 1,131,607 markers from 13,519 species and is built using 99,227 reference genomes from 16,622 species retrieved from GenBank (January 2019). Low quality alignments were removed via the filter: MAPQ <5.

Sample Rarefaction: To allow for a consistent comparison between the initial and the retest metagenomic profiles, all cleaned samples' sequences were randomly sub-sampled at 20 Million reads (post QC).

Questionnaire: Participants were asked to fill out a questionnaire that aimed at assessing the adherence to the diet recommendations and at quantifying the change in the habitual diet and body weight following the recommendations. Information gathered included:

Body weight at the start and at the end of the program

Adherence to the diet recommendations (Possible answers: No, 1-2 days per week, 3-5 days per week, 6-7 days per week; mapped to values: 0, 1, 2, 3)

Perceived level of change in the diet (Possible answers: No change, Minor change, Major change, Complete change; mapped to values: 0, 1, 2, 3)

Data Selection: Metagenomic data and questionnaire answers have been collected from 82 participants; this data set was analyzed for this Example. As the goal of this analysis is to understand the changes in the gut microbiome following, for instance, ZOE personalized diet recommendations, users that did not follow the recommendations at least 1-2 days per week were filtered out, as were users for which the diet did not change at all (Adherence >0 and Perceived level of diet change >0).

As mentioned in the methods section, to allow for a consistent comparison between the initial and the retest metagenomic profiles, all cleaned participants' sequences were sub-sampled to 20 Million reads. Users for which either of the two metagenomic datasets (initial or retest) did not reach 20 Million clean reads were filtered out, to avoid microbe abundances being influenced by a different read depth between the two time points. In total, data from 67 participants are selected and analysed in the following.

### Data Analysis

The ‘good bugs’ and the ‘bad bugs’: In this Example, the terms “15 good bugs” and “15 bad bugs” refer to the top-15 and bottom-15 species described in FIG. 6 of Asnicar et al. (Nat Med. 27:321-323, 2021; doi.org/10.1038/s41591-020-01183-8), which describes the 30 species with the strongest overall correlations with a selection of markers of nutritional and cardiometabolic health in the PREDICT study. The 15 good bugs are positively (negatively) correlated with markers associated with good (poor) health, while the 15 bad bugs are negatively (positively) correlated with markers associated with poor (good) health. The following table provides the list of 15 good bugs and 15 bad bugs.

| Good bugs | Bad bugs |
|---|---|
| Firmicutes bacterium CAG: 95 | Clostridium leptum |
| Haemophilus parainfluenzae | Ruthenibacterium lactatiformans |
| Oscillibacter sp. 57_20 | Collinsella intestinalis |
| Firmicutes bacterium CAG: 170 | Escherichia coli |
| Roseburia sp. CAG: 182 | Blautia hydrogenotrophica |
| Clostridium sp. CAG: 167 | Eggerthella lenta |
| Oscillibacter sp. PC13 | Clostridium sp. CAG: 58 |
| Eubacterium eligens | Ruminococcus gnavus |
| Prevotella copri | Clostridium spiroforme |
| Veillonella dispar | Clostridium bolteae CAG: 59 |
| Faecalibacterium prausnitzii | Clostridium innocuum |
| Veillonella infantium | Anaerotruncus colihominis |
| Bifidobacterium animalis | Clostridium symbiosum |
| Romboutsia ilealis | Clostridium bolteae |
| Veillonella atypica | Flavonifractor plautii |

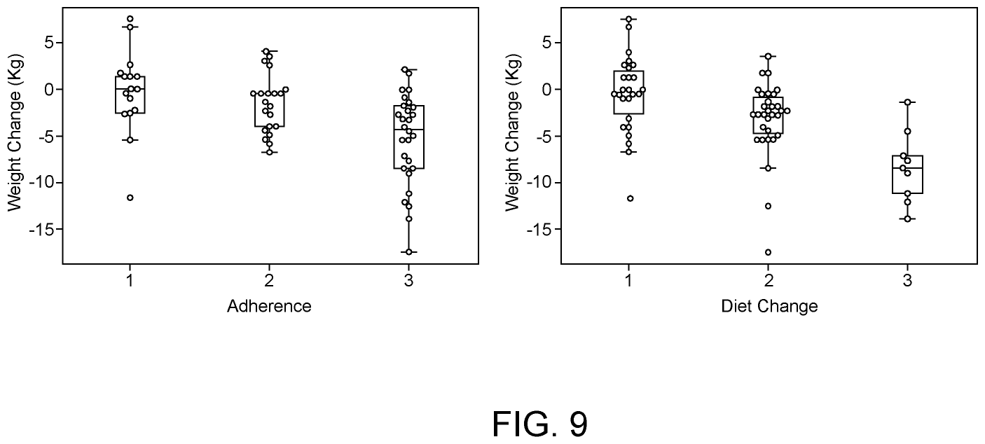

Weight Loss and Adherence: On average, the analyzed group of 67 participants lost 2.7 kg. The weight change distributions versus Adherence to the program and versus the level of diet change as consequence of the program are shown in FIG. 9. As evident from the figure, there is a clear inverse correlation between the weight change and adherence to the program: on average, the more a participant adhered to the diet recommendations the higher the weight lost. A similar correlation is observed for the level of diet change.

Statistical Analysis: The analysis considers two separate cohorts of datasets: the initial cohort that includes the metagenomic profiles from the initial test of the 67 selected participants, and the retest cohort that includes the metagenomic profiles from the re-test at the 7^{th}-9^{th} month.

The relative abundance distributions of the good and the bad bugs of the two cohorts were compared to each other using a Wilcoxon signed-rank test to establish whether there were statistically significant differences between the abundances in the two cohorts.

In order to reduce type-1 errors due to the False Discovery Rate in presence of multiple comparisons, the p-values obtained by the Wilcoxon signed-rank test were finally corrected using the Benjamini-Hochberg procedure.

### Results

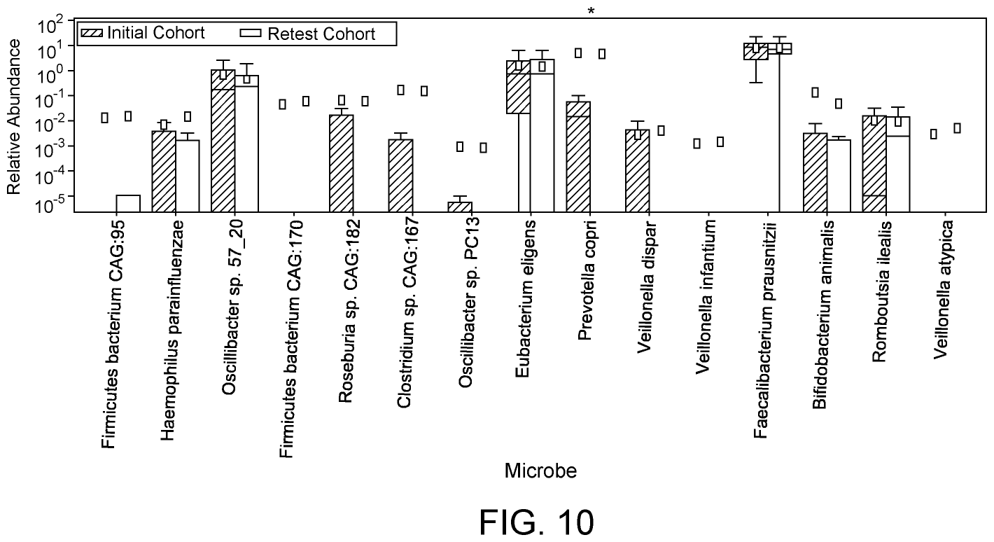

Changes in the Good Bugs: In the retest cohort, it was found that 10 good bugs over 15 have an average increase in the relative abundance compared to the abundance measured in the initial cohort. One microbe, Prevotella copri, has a statistically significant change (defined as Adjusted p-value≤0.2) in the current study, although with a small absolute size of the change (1.01 fold). The relative abundances for the initial and retest cohorts are shown in FIG. 10.

Table 2 shows the relative abundance of the 15 good bugs at the first test (Initial Abundance) and at retest time (Retest Abundance). Also shown are the Fold Change, the p-values relative to the Wilcoxon signed rank test (Wilcoxon p-value) and the p-values adjusted for False Discovery Rate (Adjusted p-value). The field Type of Change reports whether, after the diet intervention, the relative abundance increased or decreased. The table is ordered according to the Adjusted p-value.

TABLE 2

| | Initial | Retest | Fold | Wilcoxon | Adjusted | Type of |
|---|---|---|---|---|---|---|
| | Abundance | Abundance | Change | p-value | p-value | Change |
| Prevotella copri | 4.5303 | 4.5753 | 1.01 | 0.003 | 0.044 | [increased] |
| Oscillibacter sp. | 0.6281 | 0.524 | 0.834 | 0.176 | 0.59 | [decreased] |
| 57_20 | | | | | | |
| Oscillibacter sp. | 0.001 | 0.0007 | 0.723 | 0.196 | 0.59 | [decreased] |
| PC13 | | | | | | |
| Romboutsia ilealis | 0.0088 | 0.0119 | 1.354 | 0.23 | 0.62 | [increased] |
| Veillonella infantium | 0.0011 | 0.0013 | 1.145 | 0.365 | 0.72 | [increased] |
| Haemophilus | 0.0062 | 0.0149 | 2.424 | 0.454 | 0.72 | [increased] |
| parainfluenzae | | | | | | |
| Veillonella dispar | 0.0035 | 0.0041 | 1.161 | 0.456 | 0.72 | [increased] |
| Faecalibacterium | 8.3979 | 8.391 | 0.999 | 0.528 | 0.789 | [decreased] |
| prausnitzii | | | | | | |
| Firmicutes bacterium | 0.014 | 0.0147 | 1.048 | 0.601 | 0.791 | [increased] |
| CAG: 95 | | | | | | |
| Bifidobacterium | 0.137 | 0.0442 | 0.323 | 0.718 | 0.898 | [decreased] |
| animalis | | | | | | |
| Firmicutes bacterium | 0.0428 | 0.0604 | 1.412 | 0.754 | 0.904 | [increased] |
| CAG: 170 | | | | | | |
| Roseburia sp. | 0.0477 | 0.0648 | 1.357 | 0.823 | 0.909 | [increased] |
| CAG: 182 | | | | | | |
| Eubacterium eligens | 1.4463 | 1.394 | 0.964 | 0.825 | 0.909 | [decreased] |
| Clostridium sp. | 0.14 | 0.1617 | 1.155 | 0.879 | 0.909 | [increased] |
| CAG: 167 | | | | | | |
| Veillonella atypica | 0.0025 | 0.0045 | 1.759 | 0.909 | 0.909 | [increased] |

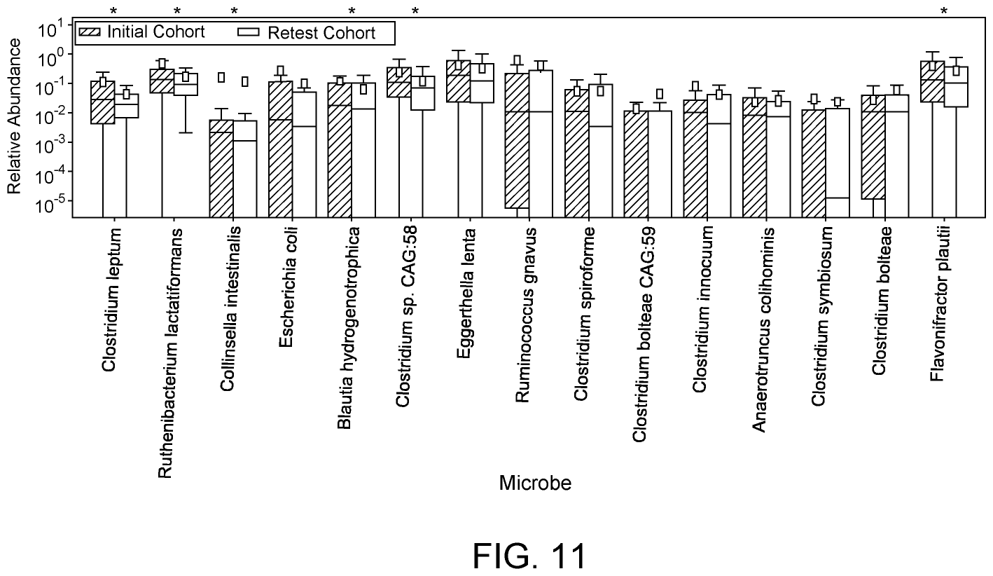

Changes in the Bad Bugs: In the retest, it was found that cohort 12 bad bugs over 15 have an average decrease in the relative abundance compared to the abundance measured in the initial cohort. For six of these microbes, the abundance changes are statistically significant (that is, the Adjusted p-value≤0.2). The relative abundances for the initial and retest cohort are shown in FIG. 11.

Table 3 shows the relative abundance of the 15 bad bugs at the first test (Initial Abundance) and at retest time (Retest Abundance). Also shown are the Fold Change, the p-values relative to the Wilcoxon signed rank test (Wilcoxon p-value) and the p-values adjusted for False Discovery Rate (Adjusted p-value). The field Type of Change reports whether after the diet intervention the relative abundance increased or decreased. The table is ordered according to the Adjusted p-value.

TABLE 3

| | Initial | Retest | Fold | Wilcoxon | Adjusted | Type of |
|---|---|---|---|---|---|---|
| | Abundance | Abundance | Change | p-value | p-value | Change |
| Clostridium sp. | 0.2108 | 0.1254 | 0.595 | 0.001 | 0.022 | [decreased] |
| CAG: 58 * | | | | | | |
| Ruthenibacterium | 0.4907 | 0.1913 | 0.39 | 0.012 | 0.074 | [decreased] |
| lactatiformans * | | | | | | |
| Clostridium leptum * | 0.1339 | 0.049 | 0.366 | 0.012 | 0.074 | [decreased] |
| Flavonifractor | 0.4187 | 0.3134 | 0.748 | 0.012 | 0.074 | [decreased] |
| plautii * | | | | | | |
| Collinsella | 0.1711 | 0.1352 | 0.79 | 0.016 | 0.081 | [decreased] |
| intestinalis * | | | | | | |
| Blautia | 0.1246 | 0.0701 | 0.563 | 0.026 | 0.112 | [decreased] |
| hydrogenotrophica * | | | | | | |
| Eggerthella lenta | 0.3986 | 0.3549 | 0.89 | 0.197 | 0.59 | [decreased] |
| Escherichia coli | 0.2853 | 0.1241 | 0.435 | 0.282 | 0.62 | [decreased] |
| Clostridium | 0.0805 | 0.0441 | 0.548 | 0.288 | 0.62 | [decreased] |
| innocuum | | | | | | |
| Anaerotruncus | 0.023 | 0.0247 | 1.076 | 0.29 | 0.62 | [increased] |
| colihominis | | | | | | |
| Clostridium | 0.0594 | 0.0592 | 0.997 | 0.403 | 0.72 | [decreased] |
| spiroforme | | | | | | |
| Clostridium bolteae | 0.029 | 0.0591 | 2.041 | 0.416 | 0.72 | [increased] |
| Clostridium bolteae | 0.0127 | 0.0433 | 3.397 | 0.552 | 0.789 | [increased] |
| CAG: 59 | | | | | | |
| Ruminococcus | 0.5978 | 0.4624 | 0.774 | 0.606 | 0.791 | [decreased] |
| gnavus | | | | | | |
| Clostridium | 0.0337 | 0.0252 | 0.747 | 0.873 | 0.909 | [decreased] |
| symbiosum | | | | | | |

* Abundance change statistically significant (Adjusted p-value ≤ 0.2).

### DISCUSSION

As shown in FIG. 9, following personalized diet recommendations results in significant weight loss (average of 2.7 kg).

In addition, adhering to the program has positively influenced the gut microbiome of participants, by modulating the relative abundance of the microbes most associated with a selection of good and poor health markers, as identified by the PREDICT Study Asnicar et al. (Nat Med. 27:321-323, 2021).

In particular, the relative abundance of microbes that are associated with good health markers (good bugs) appears to increase after the program, while conversely the relative abundance of microbes that are associated with poor health markers (bad bugs) was reduced. With the limited statistical power of the current sample size, six of the bad microbes' changes are statistically significant.

The modulation of the bad bugs in the current sampling is larger in size and statistically significant more often than that of the good bugs. This result can be explained at least in part by noticing that it is relatively easy to reduce a gut microbe abundance by changing to a diet that is unfavorable to that microbe. In contrast, increasing the abundance of a microbe is achievable only if that microbe is already present in the host: if not, to increase its abundance a diet favorable to that microbe is not enough, the host needs also to come in contact with that microbe to acquire it.

This example demonstrates that dietary intervention can be used to modify (improve) the gut health of individuals.

### Example 2: Randomized Control Trial Study Format

This example describes a study format that allows analysis of the impact of adherence to personalized diet recommendations on the abundance of “good” gut microbiome microbes and “bad” gut microbiome microbes in a large group of human subjects. Though similar to the study described in Example 1, the study described in this Example is designed as a parallel randomized at-home study aimed at improving cardiometabolic risk markers in a disease-free adult US population that is reflective of the general US phenotype. Examples of specific individual microbes and sets of microbes associated with and/or linked to poor health and others associated with and/or linked to pro-health conditions are described elsewhere in this document, though it is acknowledged that those microbe lists are not exhaustive or intended to be limiting.

In the study format described in this Example, subjects are tested throughout an initial eighteen-week period of adherence to diet recommendations, and then are further tested during follow-up period(s). An exemplar of this study format can be found at clinicaltrials.gov as “ZOE METHOD Study: Comparing Personalized vs. Generalized Nutrition Guidelines”, NCT Identifier Number NCT05273268.

In the described example, the Active Comparator (Control Arm) will receive the US government-standard guidelines for dietary advice in the form of the USDA dietary recommendations digital leaflet. The Experimental (Intervention Arm) will receive personalized dietary guidelines created by machine learning algorithms using their personal anthropometric, gut, dietary and medical information as inputs. The guidelines will be delivered in the form of a smartphone/smart device app.

Outcomes include one or more of changes in: triglyceride (TG) level, low-density lipoprotein cholesterol (LDL) level, weight, waist circumference, blood pressure (BP), glucose control (measured for instance using HbA1c, glucose, insulin, and/or C-peptide), cardiovascular risk (ApoB), microbiome, energy, and diet quality. Additional optional exploratory outcomes include skin quality, inflammation (e.g., hsCRP), mood, and/or hunger.

Study Population: U.S. adult subjects are invited to take part in a 4-month long intervention with optional follow-ups at 6-12 months. The study is powered on 300 participants in total completing the study (150 per arm); 470 people (180 per arm) will be recruited to allow up to 376 to be randomized to treatment, allowing 20% drop-out post randomization (given the remote nature of the study and the high desire to obtain the ZOE product at no cost as part of the study). The differential in participant burden (intervention arm) vs lack of intervention (reduced motivation) in control arm is likely to balance out dropout rates.

### Study Power:

Between treatment effect: at P=0.05, 2-sided, 90% power, 150 people per arm will detect the following: TG-0.21 mmol/L (SD 0.55); LDL-C-0.30 mmol/L (SD 0.8); SBP-5.27 (SD 14); DBP-3.014 (SD 8); Weight-5.7 kg (SD 15)
Within treatment effect: at P=0.05, 2-sided, 80% power, 150 people per arm will detect the following: TG-0.122 mmol/L (SD of diff 0.53), LDL-C-0.102 mmol/L (SD of differences 0.44), weight 2.9 kg (SD of difference 6.21)*ZOE re-test data used for weight

Study powered on CRESSIDA multi-component dietary intervention study PMID: 25787998, using population SD for between treatment effects and SD of paired differences (post-minus-pre-treatment) for within treatment effects.

Participants will be generally healthy, not pregnant or breastfeeding, not taking any medications that can alter glucose or lipid metabolism, fall above the 25th percentile sex—and ethnicity-specific waist circumference (self-reported), and have fruit and vegetable intake below the 75th percentile of the US population (NHANES data). As this study will be run as a sub-cohort of the PREDICT 3.1 study based within the ZOE product, participants are not existing users of the product and must not have participated in any PREDICT studies to date. Exemplary participant criteria are provided in Table 4.

TABLE 4
Eligibility Criteria

| Inclusion | Exclusion |
|---|---|
| Age 40-70 y | Have taken part in the ZOE product or any PREDICT study |
| (aim for equal | beforehand. |
| numbers within | Are unable to read and write in English, as the ZOE app is available |
| each decade | in English. |
| range) | Do not complete the first laboratory (e.g., Quest) visit successfully. |
| Males and | iPhone/Android not compatible with app. |
| females (aim | Medications affecting lipids (lipid lowering drugs e.g. statins, anti- |
| for 40:60 split, | diabetic medications, e.g. metformin and insulin), supplements |
| up to 20:80 | including fish oil (willing to come off for 4 weeks before the start of |
| acceptable) | the study, and for duration of study). |
| Waist | Have ongoing inflammatory disease i.e. rheumatoid arthritis (RA), |
| circumference | systemic lupus erythematosus (SLE), polymyalgia and other |
| greater than | connective tissue diseases. |
| ethnic-specific | Have had cancer in the last three years, excluding skin cancer. |
| 25th percentile | Have chronic gastrointestinal disorders including inflammatory |
| Fruit and Veg | bowel disease (IBD) or Coeliac disease (gluten allergy), but not |
| intake below | including irritable bowel syndrome (IBS). |
| 75th percentile | Are taking the following daily medications: immunosuppressants, |
| Willing to | corticosteroids, antibiotics in the last three months; not including |
| comply with the | inhalers. |
| study protocol | Are users of prescription PPIs (such as omeprazole and |
| Confirm they | pantoprazole), unless they are able to stop two weeks before the |
| do not meet | start of the study and remain off them for the entire duration of the |
| any exclusion | study (provided their treating physician deems it safe for them to do |
| criteria | so). |
| | Are currently suffering from acute clinically diagnosed depression or |
| | anxiety disorder. |
| | Have had a heart attack (myocardial infarction) or stroke in the last |
| | 6 months. |
| | Are pregnant or planning pregnancy in next 12 months, or are |
| | breastfeeding. |
| | Are vegan, have an eating disorder, or are unwilling to take foods |
| | that are part of the study. |
| | Have an allergy to adhesives, which would prevent proper |
| | attachment of the continuous glucose monitor. |

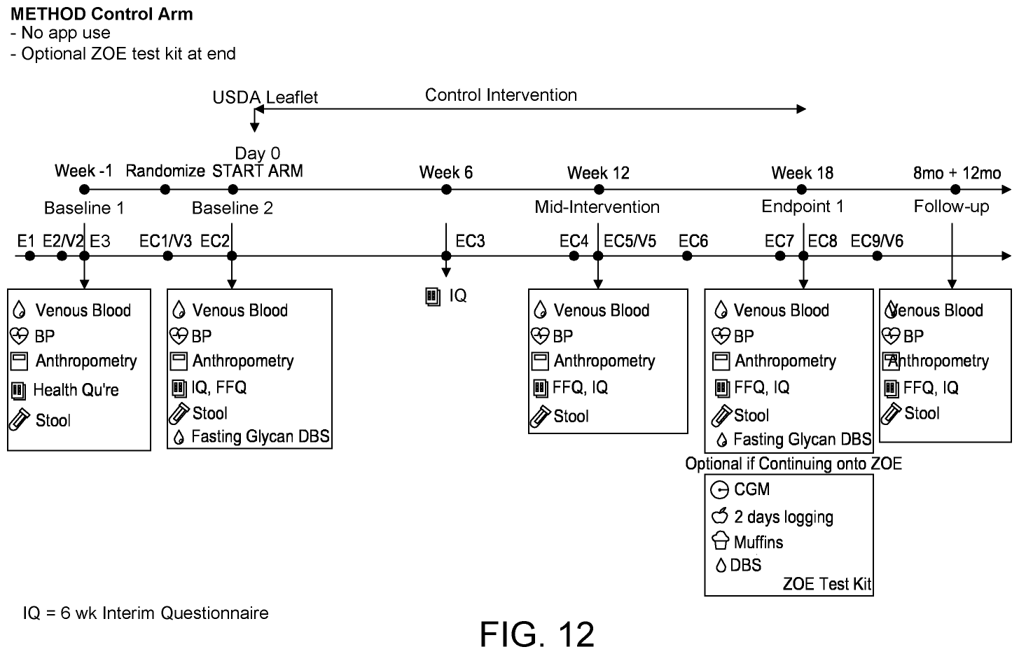

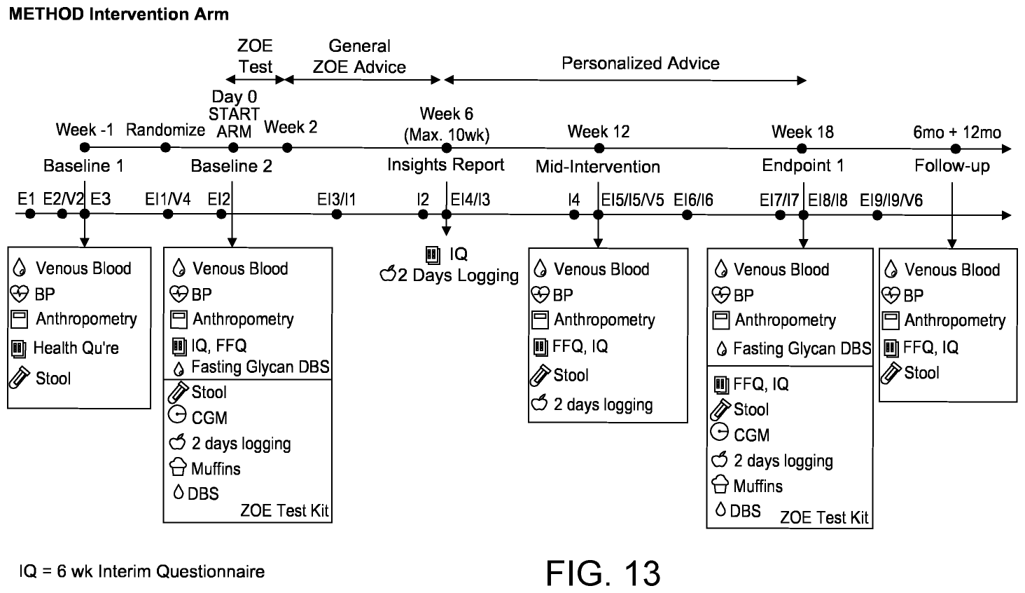

Design: The ZOE METHOD Study will take place remotely, with all study materials shipped to participants, in addition to four clinic-based blood draws at Quest Patient Centers. The study is marked by four timepoints (two at baseline, one mid-intervention, and one end-point) before two scheduled follow-ups. FIG. 12 and FIG. 13 illustrate the overall structure and elements of The ZOE METHOD Control Arm (FIG. 12) and Intervention Arm (FIG. 13) of the described Randomized Control Trial Study.

Participant Contact: Regular contact will be made with the participants via email, phone, their “application” (app) and in-app messages for the period of the treatment to encourage compliance and answer any queries.

Health history, lifestyle and habitual intake information: Participants will be asked to complete multiple questionnaires online throughout the study, including an assessment of lifestyle aspects pertaining to nutrition (exercise, sleep) and eating habits and appetite, as well as health and medical history for the assessment of physiological status and potential genetic-derived risk scores, and finally their habitual dietary intake through a food-frequency questionnaire pertaining to the previous month with portion sizes. Intervention participants will also be asked to log a weighed food diary using the ZOE app for up to 4 consecutive days per month (total of 4 logging periods) as well as a diet history questionnaire with portion sizes about the month preceding their study period.

Gut Microbiome Profiling: Gut microbiome profiling is carried out essentially as described in Example 1. Stool samples will be deposited into a DNA/RNA Shield buffer tube for stability at ambient temperature and sent by standard mail, for instance, to an analysis laboratory (such as Zymo Laboratories).

Once the sample is received at the laboratory, an unbiased DNA isolation will be performed (for instance, using ZymoBIOMICS-96 MagBead DNA Kit). The isolated microbial DNA is then prepared for shotgun metagenomics sequencing (for instance, using the Nextera DNA Flex Library Prep Kit).

Each sample will be sequenced with a depth of at least 3.75 Gb, for instance using the 150×2 chemistry on the NovaSeq platform (S4 flow cells).

The same procedure is to be repeated at each time of retesting.

Metagenome sequencing & quality control: Metagenome sequencing and quality control are carried out essentially as described in Example 1. All metagenomic sequence data will undergo a quality control analysis as implemented in the pipeline available online at github.com/SegataLab/preprocessing. For each sample, the pipeline (1) removes from low quality reads (quality score <Q20), removes too short reads (<75 bp), and reads with more than 2 ambiguous nucleotides; (2) screens and removes reads from the phi X 174 Illumina spike-in genome and human associated reads (hg19); and (3) splits and sorts the remaining cleaned reads in forward, reverse and unpaired read files.

Metagenome taxonomic profiling: The cleaned metagenome datasets are analysed, for instance by MetaPhIAn (Truong et al., Nat. Methods 12, 902-903, 2015) v.3.0 (Beghini et al., Elife. 10:e65088, 2021. doi: 10.7554/eLife.65088) or v.4.0 (Blanco-Miguez et al., bioRxiv, biorxiv.org/content/10.1101/2022.08.22.504593v1, 2022. doi.org/10.1101/2022.08.22.504593) to identify the sample taxonomic profiles and to quantify the microbe relative abundances. MetaPhIAn v.3.0 maps the metagenomes against a marker gene database, e.g., using Bowtie 2 with the parameter ‘very-sensitive’. The marker gene database consists of 1,131,607 markers from 13,519 species and is built using 99,227 reference genomes from 16,622 species retrieved from GenBank (as of 2019). Low quality alignments are removed via the filter: MAPQ <5. MetaPhIAn v.4.0 extends MetaPhIAn 3 by targeting with the same species-specific unique marker genes approach a much larger set of species that includes also species-level genome bins (SGBs, doi.org/10.1016/j.cell.2019.01.001) solely defined on metagenome-assembled genomes (MAGs). The database of MetaPhIAn v.4.0 is based on a curated collection of 1.01 M prokaryotic reference genomes and MAGs that were recapitulated in 26,970 SGBs and for which 5.1 M total unique marker genes were identified.

Sample Rarefaction: To allow for a consistent comparison between the initial and the retest metagenomic profiles, all cleaned samples' sequences may be randomly sub-sampled at 20 Million reads (post QC).

Stratified randomization: Treatment will be allocated by minimization (using MinimPy software) for the following:

| Stratifier | Category 1 | Category 2 |
|---|---|---|
| Baseline Fruit and Veg intake (cut | ≤1.56 cup eq/day (234 g) | >1.56 cup eq/day (234 g) |
| off at median adult intake; equal | | |
| numbers across intervention) | | |
| Ethnic-specific Waist Circ. | ≤ Male | >50th percentile in |
| (50th percentile) | White: 105.7 cm | Category 1 |
| | Black: 102.5 cm | |
| | Asian: 93.0 cm | |
| | Hispanic: 103.6 cm | |
| | ≤ Female | |
| | White: 98.9 cm | |
| | Black: 104 cm | |
| | Asian: 86.9 cm | |
| | Hispanic: 100 cm | |
| Sex | Female | Male |

Baseline 1: Participants will complete two baselines; the first baseline (Week-1) consists of a visit to a Quest clinic to provide a fasted blood draw, blood pressure (BP), and anthropometric measurements (waist and hip circumference, body weight) while completing food frequency, lifestyle and health/medical history questionnaires at home. BP and anthropometric measurements are completed by the participant at home as well. Participants who do not complete their first clinic visit (e.g., a Quest clinic) will be withdrawn; those who successfully complete the first baseline are randomized to the control or intervention arm.

Randomization: Participants will be assigned to either the intervention (ZOE personalized dietary advice) or control arm (USDA generalized dietary advice) once they have successfully completed Baseline 1 tasks.

Baseline 2: The second baseline will take place one week after the first (Week 0) and consists of a second visit to a clinic (e.g., a Quest clinic) to provide anthropometry, BP and a fasted blood draw, followed by another questionnaire and at-home stool sample collection for microbiome assessment. Both arms complete BP and anthropometric measurements at home again, as well as a fasted finger-prick dried blood spot test (see FIGS. 12 and 13).

Treatment: Treatment according to arm allocation starts at this second baseline; control participants are emailed a copy of the USDA Dietary Guidelines for Americans leaflet along with a short video where the dietary advice is explained. They are asked to follow this guidance.

Intervention participants (FIG. 13) receive the ZOE product and PREDICT 3.1 test kit, which they are asked to complete (thus partaking in multiple postprandial test meal challenges, providing continuous glucose measurements over 7-14 days, a post-prandial triglyceride assessment by dried blood spot collection, and logged dietary intake). While their testing results are being assimilated into a personalized dietary guidance plan, participants receive a general set of guidance through the ZOE app that incorporates brief lessons about health, diet, the gut microbiome and metabolic responses.

Personalized dietary advice (Week 6 to Week 18): Both the control (FIG. 12) and intervention (FIG. 13) groups complete a health and lifestyle questionnaire at week 6. The control group continues following the USDA leaflet advice.

Once their personalized nutritional guidance has been generated from the information collected in the testing phase (Week 0-2), the intervention group receives this personalized advice through a smart device. They are asked to complete a weighed food diary for 4 days after receiving their personalized dietary recommendations.

Mid-intervention measures are taken at Week 12. All participants complete a third clinic visit (e.g., at a Quest clinic) to provide a fasted blood sample, BP and anthropometric measurements, as well as measuring these at home again alongside completing questionnaires and a stool sample collection. The intervention group is asked to complete a weighed food diary for the third time.

Endpoint measures are taken at Week 18. Both control and intervention participants complete their fourth clinic assessment for anthropometry, BP and fasted blood collection, answer online questionnaires and provide a post-treatment stool sample, as well as completing a fasted finger-prick dried blood spot test, BP and anthropometric measures at home. The intervention group completes the ZOE product and study for the second time (FIG. 13), to assess postprandial triglyceride response and continuous glucose measurements, as well as their fourth weighed food diary.

At this endpoint, control-group participants are given the option to complete the ZOE product PREDICT 3.1 at the expense of the study, so that postprandial measurements and continuous glucose can also be assessed in this group following the control treatment.

Follow-ups: Control participants who chose to opt-in to the ZOE product at week 18 are asked to follow their resulting ZOE personalized dietary recommendations for a further 18 weeks, in a cross-over style design. These participants are followed-up at Month 8 (Week 36) and again at Month 12 with a clinical visit, questionnaires and stool sample collection. Intervention participants will undergo the same follow-ups, however the first of these will take place instead on Month 6. There is no follow up for control participants who do not opt-in to ZOE after their control phase, i.e. opt in to a cross-over arm of the trial.

Assessment of postprandial metabolic responses (Intervention group only): Participants will be provided with a standardized dietary intervention in the form of muffins, to be consumed at breakfast on two days and a subsequent lunch meal on one day. Participants are instructed to fast before and after these tests, but are free to eat and drink as they wish during the rest of the study. During this time participants will be wearing a continuous glucose monitor and will complete a 6 h finger-prick dried blood spot sample to assess triglycerides after their breakfast-lunch test. This protocol is equivalent to that followed by PREDICT 3.1 participants (NCT04735835).

Participants are expected to experience decreases in fasting triglyceride following ZOE dietary intervention.

Outcomes: The ZOE Method Study investigates the effect(s) of following personalized vs. general nutritional guidelines on certain cardiometabolic and diet-related disease risk factors, primarily including low-density-lipoprotein cholesterol (LDL-C) and triglycerides (mmol/L) assessed at each fasted blood-draw. Secondary and exploratory outcomes are listed below, and include weight, waist circumference, blood pressure (BP), glycemic control, gut microbiome assessment, and self-reported hunger.

It is expected that weight loss and an increase in diet quality will be observed in subjects who have adhered to ZOE personalized diet recommendations.

Upon assessment for species richness and diversity (for instance, using technique(s) described herein or others known in the art), it is predicted that “good bugs” in the gut microbiomes of experimental group subjects who have gone through the ZOE method will experience an average increase in relative abundance compared to that of the initial and control cohorts over the same period of time, and that bad bugs will experience an average decrease in relative abundance.

As such, this experiment is expected to demonstrate the occurrence of an overall positive change in gut health after personalized dietary intervention.

Outcome analysis optionally may be stratified according to baseline combined fruit and vegetable intake, age and sex.

TABLE 5
Outcome measures:

| | | Measured at (timepoint) | | | | | |
|---|---|---|---|---|---|---|---|
| | | | | Mid- | | | 9 & 12 |
| Outcome | Measurement | Baseline 1 | Baseline 2 | intervention | Endpoint 1 | Other | month |

Laboratory (e.g., Quest) Measurements Bloods/Blood Pressure

| | | | | | | |
|---|---|---|---|---|---|---|
| ApoA, ApoB | Fasted Venous | X | | | X | X |
| | Blood | | | | | |
| Glucose | Fasted Venous | X | X | X | X | X |
| | Blood | | | | | |
| Lipid Panel | Fasted Venous | X | X | X | X | X |
| Non-HDL and Calculated | Blood | | | | | |
| Components | | | | | | |
| total cholesterol | | | | | | |
| HDL-cholesterol | | | | | | |
| Triglycerides | | | | | | |
| Direct LDL | | | | | | |
| HbA1c | Fasted Venous | X | | | X | X |
| | Blood | | | | | |
| Insulin | Fasted Venous | X | X | X | X | X |
| | Blood | | | | | |
| C-peptide | | X | | | X | X |
| hsCRP | Fasted Venous | X | X | X | X | X |
| | Blood | | | | | |
| Liver function panel | Fasted Venous | X | | | X | X |
| Total protein | Blood | | | | | |
| Albumin | | | | | | |
| Globulin (calc'd) | | | | | | |
| Total bilirubin | | | | | | |
| Direct bilirubin | | | | | | |
| Indirect bilirubin (calc'd) | | | | | | |
| Alkaline Phosphatase | | | | | | |
| AST | | | | | | |
| ALT | | | | | | |
| TNF alpha | Fasted Venous | X | | | X | X |
| | Blood | | | | | |
| Blood Pressure | At analysis lab | X triplicate | | X | X | X |
| | & Home BP | morning, | | | | |
| | Monitor | day-time | | | | |
| | | & evening | | | | |
| | | measures | | | | |
| | | over 2 | | | | |
| | | days | | | | |
| IgG glycome, glycans | Fasted DBS | | X | | X | |
| | card | | single | | single | |

Anthropometry/Questionnaires/Stool

| | | | | | | | |
|---|---|---|---|---|---|---|---|
| Weight | At analysis lab | X | X | X | X | Every 6 | X |
| | & Home | | | | | weeks | |
| | Scales, IQ | | | | | from B2 | |
| Waist Circumference | At analysis lab | X | X | X | X | Every 6 | X |
| | & Home | | | | | weeks | |
| | Measuring | | | | | from B2 | |
| | Tape, IQ | | | | | | |
| Hip Circumference | At analysis lab | X | X | X | X | Every 6 | X |
| | & Home | | | | | weeks | |
| | Measuring | | | | | from B2 | |
| | Tape, IQ | | | | | | |
| Habitual Diet | ZOE FFQ - diet | X | | X | X | | X |
| | quality score | | | | | | |
| Eating habits and | ZOE Eating | X | | X | X | | X |
| lifestyle | Habits Lifestyle | | | | | | |
| | Questionnaire | | | | | | |
| Health Profile | ZOE Health | X | | X | X | | X |
| | Questionnaire | | | | | | |
| Microbiome | Stool Sample | X | X | X | X | | X |
| Hunger | IQ | | X | X | X | Every 6 | X |
| | | | | | | weeks | |
| | | | | | | from B2 | |
| Energy | IQ | | X | X | X | Every 6 | X |
| | | | | | | weeks | |
| | | | | | | from B2 | |
| Mood | IQ | | X | X | X | Every 6 | X |
| | | | | | | weeks | |
| | | | | | | from B2 | |
| Sleep | IQ | | X | X | X | | X |
| Skin quality | IQ | | X | X | X | | X |
| Satisfaction | | | | | X | E1 | |

Intervention arm only measures

| | | | | | |
|---|---|---|---|---|---|
| Efficacy of plan | Logged days (3 | X | X | X | X |
| | per month) | | | | |
| Efficacy of diet | Day score >60 | X | X | X | X |
| Postprandial TG | 6 h DBS, Muffin | | X | | X |
| | Challenges | | | | |
| Postprandial Glucose | Muffin | | X | | X |
| | Challenges | | | | |
| Continuous Glucose, TIR | CGM, Imputed | | X | | X |

DBS = dried blood spot; FFQ = Zoe Food Frequency Questionnaire; IQ = Zoe Interim Questionnaire.

TABLE 6
Outcome obtained by Questionnaire(s):

| Outcome | Timepoint(s) | Follow-up 6/8, 12 mo |
|---|---|---|
| Health Profile | B1 | X |
| FFQ | B1, MI, E1 | X |
| Eating habits + Lifestyle | B1, MI, E1 | X |
| Weight | B2, 6 wks, MI, E1 | (from analysis lab, e.g., Quest) |
| Waist Circ | B2, 6 wks, MI, E1 | (from analysis lab, e.g., Quest) |
| Hip Circ | B2, 6 wks, MI, E1 | (from analysis lab, e.g., Quest) |
| Sleep | B2, 6 wks, MI, E1 | (from Lifestyle Questionnaire) |
| Pittsburgh Sleep Q | | |
| Mood | B2, 6 wks, MI, E1 | X^{A} |
| Energy * | B2, 6 wks, MI, E1 | X^{A} |
| Hunger ^{§} | B2, 6 wks, MI, E1 | X^{A} |
| Skin | B1 (Health Q), 6 wks, MI, E1 | X |
| Menopause Symptom | | |
| Severity | | |
| Satisfaction | E1/Follow-up 1 Control-turned- | |
| | user | |
| Adherence | | |

B1 = Baseline 1,
MI = mid-intervention,
B2 = Baseline 2,
E1 = Endpoint 1
* Based on Ware & Gandek (J Clin Epidemiol. 51(11): 903-912, 1998)
^{§} Informed by Professor John Blundell, School of Psychology, University of Leeds
^{A}Timeframe of the standard questionnaire modified to better fit the current study

TABLE 7
Outcome obtained by ZOE Program Questionnaires
(intervention & follow-up groups):

| Question topic | Source | Text | Timepoints |
|---|---|---|---|
| Hunger | Track your biology | | Daily |
| Energy | Track your biology | | Daily |
| Bowel Habits | Track your biology | | Daily |
| Bowel Habits | Track your biology: | Bristol | Daily |
| | follow-on question | Stool Chart | |

### Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds and nucleic acid or amino acids sequences referenced herein that are available in a public database, the information in the database entry is incorporated herein by reference as of the date that the database identifier was first included in the text of an application in the priority chain.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8th Edition (Ed. J. Law & R. Rennie, Oxford University Press, 2020).

What is claimed is:

1. A method, comprising:
preparing a food guidance program for a subject;
communicating the food guidance program to the subject; wherein, when the subject follows the food guidance program:
presence or relative abundance of at least one pro-health indicator microbe in the microbiome of the subject is increased, wherein the one or more pro-health indicator microbes are selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; or
presence or relative abundance of at least one poor health indicator microbe in the microbiome of the subject is decreased, wherein the one or more poor health indicator microbe selected from the group consisting of Clostridium sp CAG 58, Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Blautia hydrogenotrophica, Anaerotruncus colihominis, Ruminococcus gnavus, Flavonifractor plautii, Clostridium leptum, Ruthenibacterium lactatiformans, and Escherichia coli;
or both.

2. Use of a diet program to improve the gut microbiome of a subject, wherein the improvement to the gut microbiome comprises:
increasing presence or relative abundance of at least one pro-health indicator microbe in the microbiome of the subject;
decreasing presence or relative abundance of at least one poor health indicator microbe in the microbiome of the subject; or
both.

3. The use of claim 2, wherein the diet program is prepared at least in part based on:
presence, absence, or relative abundance of at least one pro-health indicator microbe in the microbiome of the subject; and/or
presence, absence, or relative abundance of at least one poor health indicator microbe in the microbiome of the subject.

4. A method, comprising:
preparing a food guidance program for a subject;
communicating the food guidance program to the subject; wherein, when the subject follows the food guidance program:
presence or relative abundance of at least one pro-health indicator microbe in the microbiome of the subject is increased;
presence or relative abundance of at least one poor health indicator microbe in the microbiome of the subject is decreased;
or both.

5. A method, comprising:
detecting in a microbiome sample from a human subject one or more pro-health indicator microbes selected from the group consisting of Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and
modifying dietary intake of the human using a food guidance program that increases growth or survival of one or more of the pro-health indicator microbe(s);
and/or
detecting in a microbiome sample from the human subject one or more poor health indicator microbe selected from the group consisting of Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Clostridium sp CAG 58, Blautia hydrogenotrophica, Anaerotruncus colihominis, Ruminococcus gnavus, Flavonifractor plautii, Clostridium leptum, Ruthenibacterium lactatiformans, and Escherichia coli; and
modifying dietary intake of the human using a food guidance program that decreases growth or survival of one or more of the poor health indicator microbe(s).

6. The method of claim 1, 4, or 5, or the use of claim 2 or 3, wherein the food guidance program is personalized for the subject.

7. A method of altering abundance of at least one microbial species in a gut microbiome of a subject, comprising:
providing the subject with a personalized food guidance program, which personalized food guidance program is developed based at least in part on a food score personalized for the subject, and
modifying food intake of the subject based on the personalized food guidance plan, thereby altering the abundance of at least one microbial species in the gut microbiome of the subject.

8. The method of claim 7, wherein altering abundance comprises:
increasing presence or relative abundance of at least one pro-health indicator microbe in the microbiome of the subject;
decreasing presence or relative abundance of at least one poor health indicator microbe in the microbiome of the subject; or
both.

9. A method of improving gut microbiome balance/health of a subject, comprising
developing a personalized food guidance program for the subject,
communicating the personalized food guidance program to the subject, and
modifying food intake of the subject, in accordance with the personalized food guidance program, thereby improving gut microbiome balance/health of the subject.

10. A method of improving a gut microbiome profile of a subject, comprising:
assaying the gut microbiome of the subject, thereby producing a first microbiome signature of the subject;
developing a personalized food guidance program for the subject, which personalized food guidance program will improve the gut microbiome of the subject when the subject follows the program;
communicating the personalized food guidance program to the subject;
assaying the gut microbiome of the subject at a time the after the subject begins to follow the customized diet guidance plan, thereby producing a second microbiome signature of the subject;
comparing the first microbiome signature of the subject to the second microbiome signature of the subject,
wherein the first and second microbiome signatures comprise the presence, absence, or relative abundance of one or more pro-health indicator microbes and/or the presence, absence, or relative abundance of one or more poor health indicator microbes; and
wherein an increase in the presence or relative abundance of the one or more pro-health indicator microbes and/or a decrease in the presence or relative abundance of the one or more poor health indicator microbes from the first signature to the second signature constitutes an improved gut microbiome profile of the subject.

11. The use or method of claim 6, or the method of any of claims 7-10, wherein the personalized food guidance program is based at least based in part on one or more of:
a non-microbial biomarker of the subject;
a nutritional response of the subject;
medical history of the subject;
a health condition of the subject;
predicted hunger of the subject;
predicted response to food consumption of the subject;
glucose response of the subject;
fat response of the subject;
microbiome data of the subject;
data about the subject's overall health; and/or
potential health risks for the subject.

12. The use or method of any one of claim 2-4, 8, 10, or 11, wherein:
the pro-health indicator microbe is selected from the group including Prevotella copri, Blastocystis spp., Haemophilus parainfluenzae, Firmicutes bacterium CAG 95, Bifidobacterium animalis, Oscillibacter sp 57 20, Roseburia sp CAG 182, Veillonella dispar, Eubacterium eligens, Firmicutes bacterium CAG 170, Rothia mucilaginosa, Veillonella infantium, Roseburia hominis, Oscillibacter sp PC13, Clostridium sp CAG 167, Ruminococcaceae bacterium D5, Paraprevotella xylaniphila, Faecalibacterium prausnitzii, Romboutsia ilealis, and Veillonella atypica; and/or
the poor health indicator microbe is selected from the group including Clostridium sp CAG 58, Eubacterium ventriosum, Roseburia inulinivorans, Clostridium spiroforme, Clostridium bolteae CAG 59, Eggerthella lenta, Clostridium bolteae, Collinsella intestinalis, Clostridium innocuum, Blautia obeum, Clostridium symbiosum, Blautia hydrogenotrophica, Anaerotruncus colihominis, Ruminococcus gnavus, Flavonifractor plautii, Clostridium leptum, Ruthenibacterium lactatiformans, and Escherichia coli

13. The use or method of claim 1 or claim 12, wherein the pro-health indicator microbe comprises Prevotella copri.

14. The use or method of claim 1, 12 or 13, wherein the poor health indicator microbe comprises one or more of Clostridium sp CAG 58, Collinsella intestinalis, Blautia hydrogenotrophica, Flavonifractor plautii, Clostridium leptum, and/or Ruthenibacterium lactatiformans.

15. The use or the method of any one of claims 1-14, wherein the food guidance program is developed based at least in part based on or in reference to a database comprising one or more of:
correlations between food consumed and microbiome members for thousands of individual subjects; and/or
a plurality of foods designated as “to be promoted in a diet”, wherein this designation indicates the food tends to support a healthy condition in the subject, or tends to move a subject incorporating the food in their diet more toward a healthy state; and/or
a plurality of foods designated as “to be demoted in a diet”, wherein this designation indicates the food tends to not support a healthy condition, or tends to support an unhealthy condition in the subject, or tends to move a subject incorporating the food in their diet more toward a less healthy state.

16. Use of a diet program, such as a personalized diet program, to improve the gut microbiome of a subject or to alter the abundance of at least one microbial species in a gut microbiome of a subject, essentially as disclosed herein.
